(19) United States
(12) Patent Application Publication
(10) Pub. No.: US 2025/0008903 A1
(43) Pub. Date: Jan. 9, 2025

(54) MODIFIED TOM2A GENE INVOLVED IN TOBAMOVIRUS RESISTANCE

(71) Applicant: RIJK ZWAAN ZAADTEELT EN ZAADHANDEL B.V., De Lier (NL)

(72) Inventors: Jonathan KALISVAART, De Lier (NL); Daniël Johannes Wilhelmus LUDEKING, De Lier (NL); Alwin Johannes Marinus ROOVERS, De Lier (NL); Cornelis Maria Petrus VAN DUN, De Lier (NL); Ellen Anna Johanna BASTIAANSSEN, De Lier (NL); Lena Johanna HUIJBREGTS-DOORDUIN, De Lier (NL)

(21) Appl. No.: 18/774,411

(22) Filed: Jul. 16, 2024

Related U.S. Application Data
(63) Continuation-in-part of application No. PCT/EP2023/051031, filed on Jan. 17, 2023.

(30) Foreign Application Priority Data
Jan. 17, 2022 (WO) PCT/EP2022/050893
May 31, 2022 (WO) PCT/EP2022/064821

(51) Int. Cl. A01H 1/00; A01H 1/04; C12Q 1/6895

(52) U.S. Cl. CPC A01H 1/126; A01H 1/045; C12Q 1/6895; C12Q 2600/13

(57) ABSTRACT

The present invention relates to a plant comprising a modified Tom2a gene, the wildtype of which comprises SEQ ID No. 1, SEQ ID No. 2, SEQ ID No. 3, SEQ ID No. 4, SEQ ID No. 5, or SEQ ID No. 6, or comprises another homologous sequence having at least 70% sequence identity to SEQ ID No. 1, which modified gene leads to increased resistance to a Tobamovirus. The modification in the Tom2a gene leads to a modified Tom2a protein that has a deletion, a substitution, or an insertion of at least one amino acid when compared to SEQ ID No. 7, SEQ ID No. 8, SEQ ID No. 9, SEQ ID No. 10, SEQ ID No. 11, or SEQ ID No. 12, or compared to another homologous sequence having at least 70% sequence similarity to SEQ ID No. 7. The plant is preferably a plant of the family Cucurbitaceae or Solanaceae. The invention further relates to a modified Tom2a gene encoding a Tom2a protein comprising a deletion, a substitution, or an insertion of at least one amino acid when compared to SEQ ID No. 7, SEQ ID No. 8, SEQ ID No. 9, SEQ ID No. 10, SEQ ID No. 11, or SEQ ID No. 12, or compared to another homologous sequence having at least 70% sequence similarity to SEQ ID No. 7.

### RELATED APPLICATIONS AND INCORPORATION BY REFERENCE

This application is a continuation-in-part of copending International Patent Application No. PCT/EP2023/051031, filed Jan. 17, 2023, and which published as International Publication No. WO 2023/135335 on Jul. 20, 2023 and which claims priority to International Patent Application Nos. PCT/EP2022/050893, filed Jan. 17, 2022, and PCT/EP2022/064821, filed May 31, 2022, the contents of which are incorporated by reference herein in their entirety.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy, was created on Apr. 14, 2023, is named Y7954-00614SL.xml, and is 39,150 bytes in size.

### BACKGROUND OF THE INVENTION

The present invention relates to a plant comprising a modified gene which leads to resistance against a Tobamovirus. The invention further relates to methods for producing such a plant, methods for identification of the modified gene, and methods for selection of such a plant. The invention also relates to the modified gene, and the use of said gene to confer or increase Tobamovirus resistance to a plant. The invention is also directed to a marker for identification of a modified gene in a plant, and to use of said marker.

Wherever agriculture is practiced, viral diseases pose one of the major threats that vegetable growers are facing, both in protected as well as open field cultivation. Initial infection can occur through the use of infected seed, or by mechanical or insect transmission. Once a crop is infected, spread of the virus can occur rapidly by mechanical sap transmission through the use of tools and cultivation practices, or through hard-to-control vectors such as insects.

Viruses belonging to the genus Tobamovirus are often seed-bome, and are thereafter mostly spread by mechanical transmission; insects are not particularly involved in the transmission of Tobamoviruses. Common symptoms of Tobamovirus infection include yellowing through leaf chlorosis, severe or mild mosaic, leaf distortion, and fruit symptoms. Tobamoviruses are positive-strand RNA viruses; their replication takes place in highly specialized replication compartments, that are formed as spherules or vesicles in certain membranes. Viral positive-strand RNA that is synthesized within these compartments, whereby negative-strand RNA is used as a template, is released into the cytoplasm through a narrow channel that connects the inner space of the compartment to the cytoplasm. In a standard situation, the existence of this very narrow channel appears to prevent contact between the components that are involved in viral synthesis, and antiviral defense mechanisms that are available to the host plant.

Numerous genes have been recognized for their involvement in virus resistance in plants, for example because a gene is known to be part of the viral replication process that the virus requires to infect its host. Virus resistance can be based on various mechanisms, and many different phases of plant development, along with different plant defense pathways, can be involved. However, even when a gene is known to be part of the viral replication mechanism, it is not necessarily clear how that gene should or could be modified to ensure its original role is lost to the virus.

It is an object of the present invention to provide a plant showing resistance against a virus belonging to the genus of Tobamoviruses, in particular to Cucumber Green Mottle Mosaic Virus (CGMMV) or Tomato Brown Rugose Fruit Virus (ToBRFV).

The search for new genetic resources for virus resistance is permanently ongoing in plant breeding research. Developing new varieties that help the growers cope with the ever-present disease challenges requires constant adaptation to new or stronger virus variants. Research leading to the present invention focused on identifying and developing new genetics for resistance to Tobamoviruses, which affect various crops, in particular of the Cucurbitaceae and Solanaceae families. In cucumber (Cucumis sativus), the Tobamovirus species CGMMV is an important disease, and even though resistance is present, it is not absolute and stacking of various resistant backgrounds is a common practice. In the search for new resistance, a large EMS population was developed, and various mutants in a number of genes were identified by applying a TILLING approach. Mutations resulting in stop mutants, splice sites, and amino acid changes that were predicted to be deleterious when a SIFT prediction was applied, were selected.

Tom2a was one of the genes for which a number of potentially interesting EMS TILLING mutants were obtained (Example 1). Two mutants resulted in a premature stop codon, which in turn generated a truncated Tom2a protein that missed a number of domains thought to be essential for proper functioning of the protein. In addition, four mutants resulting in amino acid changes that were predicted to be deleterious, i.e. it could be expected that the function of the protein would be affected, were identified. The stop mutants were anticipated to be especially interesting, since at least part of the crucial C-terminal tail of the protein was missing. One of those, the mutant resulting in a Q268stop mutation in the encoded cucumber Tom2a protein, was phenotyped first (Example 2), to get an idea of the potential of Tom2a mutants for virus resistance in general.

In co-pending application PCT/EP2021/070104 the identification of several modifications in the Tom2a gene of Solanum pimpinellifolium is described. PCT/EP2021/070104 discloses a GGC312-314del modification, an A559G substitution, a G673A substitution, and an A844G substitution in the Tom2a gene of Solanum lycopersicum. Transfer of these modifications to Solanum lycopersicum is described, and it is shown that the presence of one or more of these modifications in the Tom2a gene in S. lycopersicum leads to increased ToBRFV resistance. The combination of the findings of CGMMV resistance due to a Tom2a modification in cucumber, and ToBRFV resistance due to a Tom2a modification in tomato, is considered to form a good basis for extrapolation of the presence of these or similar modifications to the homologous Tom2a genes of other crops, in particular crops of the Cucurbitaceae, related to cucumber, and of the Solanaceae, related to tomato, to achieve increased Tobamovirus resistance, in particular CGMMV in Cucurbitaceae or ToBRFV resistance in Solanaceae.

### SUMMARY OF THE INVENTION

According to one aspect of the present invention, the present invention relates to a plant comprising a modified Tom2a gene, the wildtype of which comprises SEQ ID No. 1, SEQ ID No. 2, SEQ ID No. 3, SEQ ID No. 4, SEQ ID No. 5, or SEQ ID No. 6, or comprises another homologous sequence having at least 70% sequence identity to SEQ ID No. 1, which modified gene leads to increased resistance to a Tobamovirus.

In some embodiments, the plant comprises a modification in the Tom2a gene that leads to a modified Tom2a protein that has a deletion, a substitution, or an insertion of at least one amino acid when compared to SEQ ID No. 7, SEQ ID No. 8, SEQ ID No. 9, SEQ ID No. 10, SEQ ID No. 11, or SEQ ID No. 12, or compared to another homologous sequence having at least 70% sequence similarity to SEQ ID No. 7. In some embodiments, the modification results in a premature stop codon which leads to a truncated protein.

In some embodiments, the Tom2a protein encoded by the modified Tom2a gene is absent, has a changed function, a reduced function, or is non-functional.

In some embodiments, the modification in the Tom2a protein is present in a transmembrane domain or in the C-terminal tail.

In some embodiments, the modification in the Tom2a protein is present in Non-CP EC1 or Non-CP EC2.

In some embodiments, the modified Tom2a protein is truncated and comprises only amino acids 1-267 of SEQ ID No. 7 or less, or the corresponding number of amino acids of a homologous Tom2a protein sequence, or the modified Tom2a protein comprises a modification in amino acids 268-279 of SEQ ID No. 7 that leads to a changed function, a reduced function, or a non-functional protein, or in the corresponding amino acids of a homologous Tom2a protein sequence, in particular of SEQ ID No. 8, SEQ ID No. 9, SEQ ID No. 10, SEQ ID No. 11, or SEQ ID No. 12.

In some embodiments, the modified Tom2a protein comprises SEQ ID No. 14.

In some embodiments, the plant is part of the family Cucurbitaceae or Solanaceae. In some embodiments, the plant is a plant of the species Cucumis sativus, Cucumis melo, Citrullus lanatus, Cucurbita pepo, Solanum lycopersicum, or Capsicum annuum, preferably a plant of the species Cucumis sativus or Solanum lycopersicum.

In some embodiments, the plant is of the family Cucurbitaceae and has increased resistance to CGMMV.

In some embodiments, the plant is of the family Solanacae and has increased resistance to ToBRFV.

According to another aspect of the present invention, the present invention provides for a modified Tom2a gene encoding a Tom2a protein comprising a deletion, a substitution, or an insertion of at least one amino acid when compared to SEQ ID No. 7, SEQ ID No. 8, SEQ ID No. 9, SEQ ID No. 10, SEQ ID No. 11, or SEQ ID No. 12, or compared to another homologous sequence having at least 70% sequence similarity to SEQ ID No. 7.

In some embodiments, the modified Tom2a gene comprises a premature stop codon and which encodes a truncated Tom2a protein

In some embodiments, the Tom2a protein encoded by the modified Tom2a gene is absent, has a changed function, a reduced function, or is non-functional.

In some embodiments, the encoded modified Tom2a protein is truncated and comprises only amino acids 1-267 of SEQ ID No. 7 or less, or the corresponding number of amino acids of a homologous Tom2a protein sequence, or the modified Tom2a protein comprises a modification in amino acids 268-279 of SEQ ID No. 7 that leads to a changed function, a reduced function, or a non-functional protein, or in the corresponding amino acids of a homologous Tom2a protein sequence, in particular of SEQ ID No. 8, SEQ ID No. 9, SEQ ID No. 10, SEQ ID No. 11, or SEQ ID No. 12.

In some embodiments, the presence of the modified Tom2a gene in a plant leads to increased Tobamovirus resistance, in particular to increased CGMMV resistance in a plant of the family Cucurbitaceae, or to increased ToBRFV resistance in a plant of the family Solanaceae.

According to another aspect of the present invention, the present invention provides for seed, comprising the modified Tom2a gene as described herein, wherein the plant grown from the seed is a plant as described herein.

According to another aspect of the present invention, the present invention provides for a marker and use of the marker for identification of a modified Tom2a gene that leads to increased Tobamovirus resistance, in particular a marker comprising any one of SEQ ID Nos. 15-24. In some embodiments, the marker may be used for identification of a plant having a modified Tom2a gene. In some embodiments, the marker may be used for identification of increased Tobamovirus resistance in a plant.

According to another aspect of the present invention, the present invention provides for a method for increasing Tobamovirus resistance in a plant, in particular CGMMV or ToBRFV resistance, comprising introducing a modification in a Tom2a gene, wherein the modification leads to a Tom2a gene as described herein.

In some embodiments, the method for increasing Tobamovirus resistance in a plant, comprises introducing a modified Tom2a gene as described herein into a plant.

The present invention also provides for a method for producing a plant of the family Cucurbitaceae or Solanaceae that has increased Tobamovirus resistance, in particular increased CGMMV or ToBRFV resistance, comprising introducing a modification in a Tom2a gene comprising SEQ ID No. 1, SEQ ID No. 2, SEQ ID No. 3, SEQ ID No. 4, SEQ ID No. 5, or SEQ ID No. 6, or in another homologous sequence having at least 70% sequence identity to SEQ ID No. 1.

In some embodiments, the modification introduced by the method leads to a Tom2a gene as described herein.

In some embodiments, the method for the production of a plant which has increased resistance to a Tobamovirus, in particular to CGMMV or ToBRFV, comprises:
a) crossing a plant as described herein, with another plant;
b) optionally performing one or more rounds of selfing and/or crossing of the plant resulting from the cross of step a) to obtain a further generation population;
c) selecting from the population resulting from the cross of step a), or from the further generation population of step b), a plant that comprises the modified Tom2a gene as described herein.

The present invention also provides for a method for the production of a hybrid seed, comprising crossing a first parent plant with a second parent plant and harvesting the resultant hybrid seed, wherein the first parent plant and/or the second parent plant is a plant as described herein, and wherein the hybrid plant that is grown from the seed has increased Tobamovirus resistance, in particular increased CGMMV or ToBRFV resistance.

In some embodiments, a hybrid seed can be produced by the method. In some embodiments, a hybrid Cucumis sativus seed that has increased CGMMV resistance can be produced from the method. In some embodiments, a hybrid Solanum lycopersicum seed that has increased ToBRFV resistance can be produced from the method.

The present invention also provides for a method for identification of a plant comprising a modified Tom2a gene as described herein, wherein the identification comprises determining the presence of a modification in a Tom2a gene comprising SEQ ID No. 1, SEQ ID No. 2, SEQ ID No. 3, SEQ ID No. 4, SEQ ID No. 5, SEQ ID No. 6, or in another homologous sequence of SEQ ID No. 1, and optionally analyzing if the plant comprising the modification has increased resistance to a Tobamovirus, in particular to CGMMV or ToBRFV.

The present invention also provides for propagation material suitable for producing a plant as described herein, wherein the propagation material is suitable for sexual reproduction, and is in particular selected from a microspore, pollen, an ovary, an ovule, an embryo sac, and an egg cell; or is suitable for vegetative reproduction, and is in particular selected from a cutting, a root, a stem cell, and a protoplast; or is suitable for tissue culture of regenerable cells, and is in particular selected from a leaf, pollen, an embryo, a cotyledon, a hypocotyl, a meristematic cell, a root, a root tip, an anther, a flower, a seed, and a stem; wherein the plant produced from the propagation material comprises the modified Tom2a gene as described herein that provides increased resistance to a Tobamovirus, in particular to CGMMV or ToBRFV.

The present invention also provides for a method for seed production, comprising growing a plant from a seed as described herein that comprises the modified Tom2a gene as described herein, allowing the plant to produce a fruit with seed, harvesting the fruit, and extracting those seed.

### BRIEF DESCRIPTION OF THE DRAWINGS

FIG. 1—CDS sequences of wildtype Tom2a genes of Cucumis sativus (SEQ ID No. 1), Cucumis melo (SEQ ID No. 2), Citrullus lanatus (SEQ ID No. 3), Cucurbita pepo (SEQ ID No. 4), Solanum lycopersicum (SEQ ID No. 5), Capsicum annuum (SEQ ID No. 6), and alignment of SEQ ID Nos. 1-6.

FIG. 2—protein sequences of wildtype Tom2a genes of Cucumis sativus (SEQ ID No. 7), Cucumis melo (SEQ ID No. 8), Citrullus lanatus (SEQ ID No. 9), Cucurbita pepo (SEQ ID No. 10), Solanum lycopersicum (SEQ ID No. 11), Capsicum annuum (SEQ ID No. 12), and alignment of SEQ ID Nos. 7-12.

FIG. 3—Modified CsTom2a CDS (SEQ ID No. 13) and protein (SEQ ID No. 14) sequence leading to CGMMV resistance.

FIG. 4—prediction of the transmembrane protein sections and domains of the Tom2a protein of a. Cucumis sativus and b. Solanum lycopersicum.

FIG. 5—Nucleotide sequences representing marker sequences SEQ ID Nos. 15-24.

FIG. 6—Phenotyping results of Tom2a knockout mutants in S. lycopersicum.

FIG. 7—Virus titer measurements of Tom2a knockout mutants in S. lycopersicum.

### DETAILED DESCRIPTION OF THE INVENTION

The present invention provides a plant comprising a modified Tom2a gene, which modified Tom2a gene leads to increased resistance against a Tobamovirus. The presence of the modified Tom2a gene in particular leads to increased Tobamovirus resistance in a plant belonging to the family Solanaceae or in a plant belonging to the family Cucurbitaceae. The plant comprising the modified Tom2a gene is preferably a plant of the Cucurbitaceae family or a plant of the Solanaceae family, in particular a plant of the species Cucumis sativus, Cucumis melo, Citrullus lanatus, Cucurbita pepo, Solanum lycopersicum, or Capsicum annuum. A plant of the invention comprising a modified Tom2a gene is most preferably a plant of the species Cucumis sativus or of the species Solanum lycopersicum. A plant of the invention is preferably a cultivated plant, which is non-wild and has agronomical value, and is in particular agronomically elite. The presence of the modified Tom2a gene preferably leads to increased resistance against Cucumber Green Mottle Mosaic Virus (CGMMV) or Tomato Brown Rugose Fruit Virus (ToBRFV), optionally in combination with resistance against another virus, in particular another Tobamovirus. Resistance to CGMMV is preferably increased in a plant of the Cucurbitaceae family, in particular in Cucumis sativus. Resistance to ToBRFV is preferably increased in a plant of the Solanaceae family, in particular in Solanum lycopersicum.

As used herein, a Tobamovirus is in particular Cucumber Green Mottle Mosaic Virus (CGMMV) or Tomato Brown Rugose Fruit Virus (ToBRFV), but optionally also includes any other virus belonging to the Tobamoviruses. Other viruses belonging to the Tobamoviruses are for example Pepper Mild Mottle Virus (PMMoV) in Capsicum annuum, TMV, ToMV, ToMMV, BPMoV, TMGMV, PaMMV, WGMMV, ZGMMV, and KGMMV.

Phenotyping of a cucumber plant having a modified Tom2a gene for increased CGMMV resistance is performed by sowing seeds of an accession to be tested in a standard seedling tray at 23° C. After 5 days, at least 10 seedlings are transplanted to a larger pot. One week after sowing, the transplants are inoculated and transferred to a temperature regime of 20° C. by day and 18° C. by night. Inoculum is prepared by grounding leaves of cucumber plants that were infected with CGMMV in a 0.01 M phosphate buffer (pH 7.0). The seedlings are then dusted with carborundum powder prior to gently rubbing the leaf with inoculum. Resistance is scored on a scale of 1-5; the description of the scales of the scores can be found in Table 1. Observation of the symptoms on the young cucumber plants in the bio-assay is done 14-21 days after inoculation (dai). A susceptible control that does not comprise a modified Tom2a gene has to be included, an example of which is standard variety Ventura F1. A test is properly performed when a susceptible (S) control, in particular Ventura F1, has an average score that is higher than 4.9, preferably 5.0. Once this average is reached, it is a proper moment to score the assay. As is a standard prerequisite for all experiments, a test is adequately performed when plants are tested in at least 2 repetitions.

As used herein, a plant that has increased CGMMV resistance due to the presence of a modified Tom2a gene has an average score lower than 4.0, preferably an average score lower than 3.5, more preferably an average score lower than 3.0, most preferably an average score lower than 2.8, when scoring according to Table 1 is used.

TABLE 1
scales CGMMV resistance scores

| Score | Symptoms |
|---|---|
| 1 | No symptoms |
| 2 | Healthy but not clean, some small spots or discolored patches |
| 3 | Clear visible symptoms, minor in severity. Some mosaic or |
| | blistering, no deformation |
| 4 | Severe mosaic and leaf mottling in old and young leaves; starting |
| | deformation in the head |
| 5 | Severe mosaic and mottling; plants are strongly deformed |

Phenotyping of ToBRFV resistance is performed by sowing seeds of the accessions to be tested in standard seedling trays. After 14-21 days, seedlings are transplanted into larger pots. Preferably at least 10 seedlings, optimally at least 20 seedlings, are inoculated 4 weeks after sowing. Inoculum is prepared by grounding leaves of tomato plants that were infected with ToBRFV in a 0.01 M phosphate buffer (pH 7.0) mixed with celite. The seedlings are then dusted with carborundum powder prior to gently rubbing the leaf with inoculum. Resistance is scored on a scale of 0-5; the description of the scales of the scores can be found in Table 7. Observation of the symptoms on the young tomato plants in the bio-assay is done 14-21 days after inoculation (dai).

ToBRFV resistance is determined by comparison to a control variety known to be ToBRFV susceptible. Examples of ToBRFV susceptible tomato varieties that do not have the resistance conferring Tom2a allele of the invention on chromosome 8, and can therefore be used as susceptible control, are Livento F1, Adventure F1, or Eclipse F1. The test is performed with at least 10 plants of a certain line, and the average score is taken. The test is performed properly when the susceptible (S) control has an average score that is higher than 3.0, preferably higher than 3.5. Once this average is reached is a correct moment to score the assay.

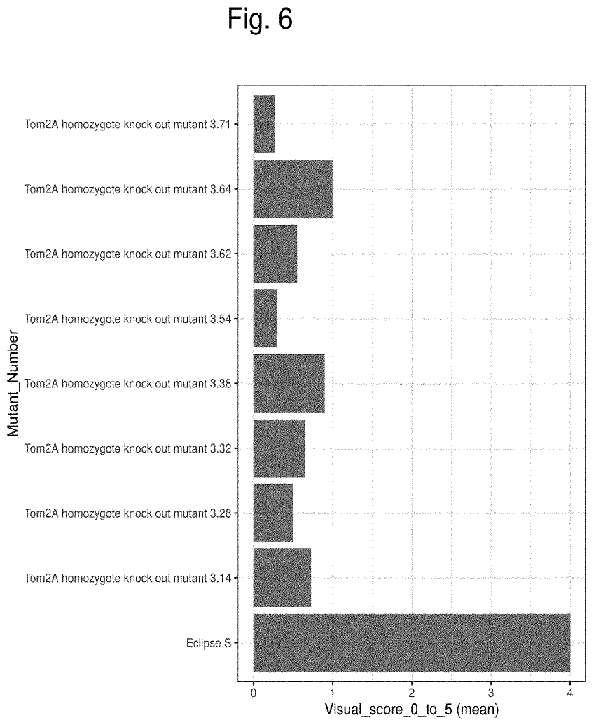

As used herein, a ToBRFV resistant tomato plant homozygously comprising the resistance conferring Tom2a allele of the invention on chromosome 8 has an average score of 1.5 or lower than 1.5, preferably a score lower than 1.0, when scoring according to Table 7 is used (Example 6, FIG. 6).

As used herein, ToBRFV resistance means that replication of the virus is reduced or absent in a plant that is infected with ToBRFV. Reduction of virus replication can be measured by a qPCR test. To determine if a line has reduction or absence of ToBRFV virus replication, the virus titer is determined in leaf samples which are taken from at least 5 plants of that line that are ToBRFV infected. From each plant a leaf punch of 6 mm in diameter is taken and subsequently ground in 500 μl of PBS buffer solution. 50 μl of the resulting suspension is used in a 96-well KingFisher Flex isolation protocol, whereby isolation of the leaf material is done using the innuPREP DNA/RNA virus PLUS Kit. The samples are then analysed in a 96CFX qPCR thermocycler (Biorad) to get a Cq_ToBRFV value, which represents the number of cycles needed to obtain the virus PCR product, using a programme of 5 minutes on 50° C. and 20 sec. on 95° C., followed by 40 cycles of 10 sec. on 95° C. and 60 sec. on 60° C.

To be able to compare the values of samples of different sizes and backgrounds, the S. lycopersicum PHD reference gene, a tomato housekeeping gene, is included in the qPCR assay, which corrects any variation in the amount of sample material, and then yields a Cq_PHD value. To accurately determine the final value for virus titer the Delta Cq method is used, with PHD as a housekeeping gene and ToBRFV as the gene of interest. The final value is the Cq_corr, which is calculated as Cq_ToBRFV−Cq_PHD. A plant is determined to have a reduction of ToBRFV virus replication when the average Cq_corr, of at least 5 plant samples, is higher than −11.00, or when the average Cq-corr is at least 5.00 higher than the average Cq_corr of a susceptible control.

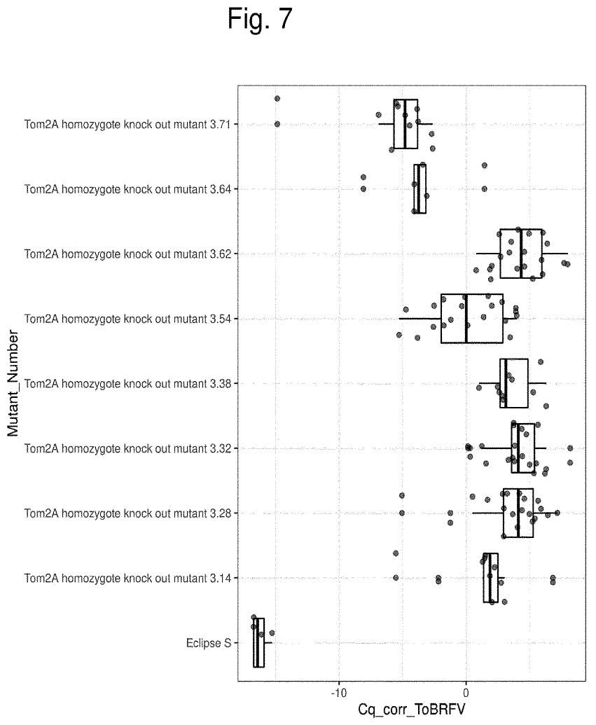

In one embodiment a ToBRFV resistant S. lycopersicum plant of the invention comprises a knockout of the Tom2a gene of the invention on chromosome 8 and has an average Cq_corr score that is in order of increased preference higher than −6.00, −5.50, −5.00, −4.50, −4.00, −3.50, −3.00, −2.50, −2.00, −1.50, −1.00, −0.50, or higher than 0.00. The knockout of the Tom2a gene leads to a loss-of-function, wherein the encoded protein is absent, has a reduced function, or is non-functional. The average Cq_corr score is preferably taken from at least 20 plants comprising the same knockout event (Example 6, FIG. 7).

A plant of the invention comprises a modified Tom2a gene homozygously or heterozygously, i.e. a modified Tom2a gene can be present on both chromosomes of a chromosome pair in the genome of a plant, or on only one chromosome of a chromosome pair. A plant of the invention comprises a plant of an inbred line, an F1 cross, a hybrid variety, an open pollinated variety, a doubled haploid, or a plant of a segregating population.

A plant of the invention that has increased Tobamovirus resistance due to the presence of a modified Tom2a gene does not require the presence of a Tobamovirus-resistance-conferring allele of a Tm-1 gene, TOM1 gene, or TOM3 gene to show resistance.

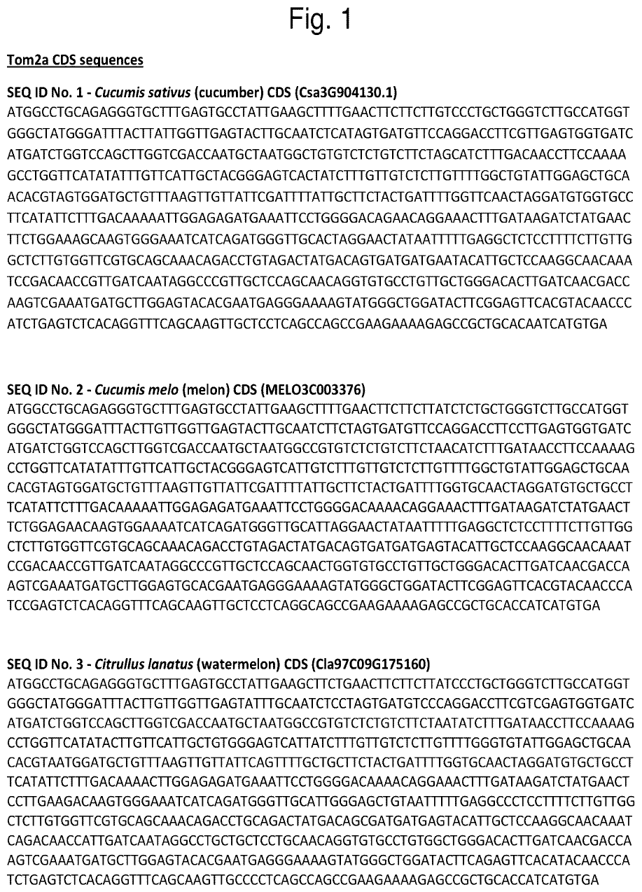

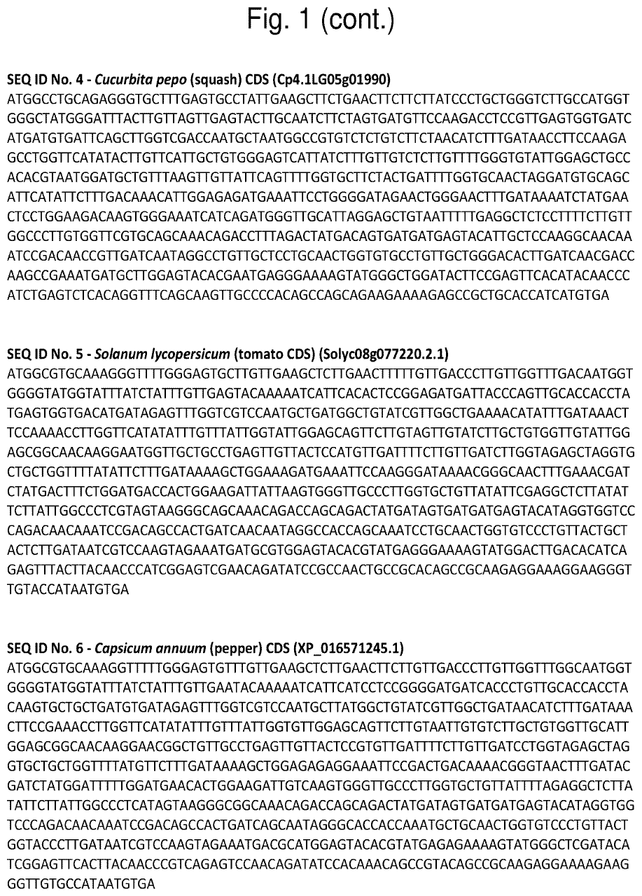

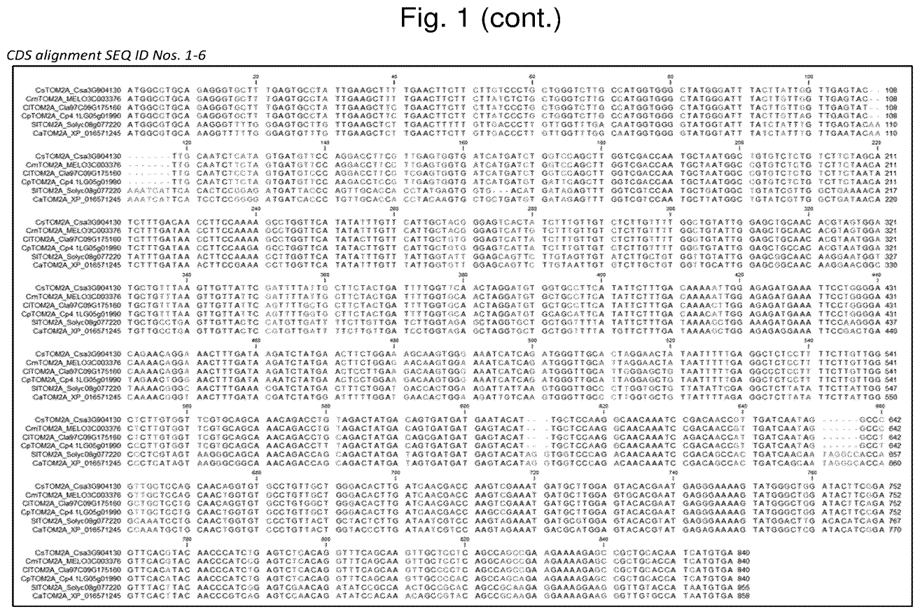

The wildtype CDS sequence of the Tom2a gene of the invention comprises SEQ ID No. 1 for Cucumis sativus, or comprises a homologous sequence of a Tom2a gene in another crop having at least 70% sequence identity to SEQ ID No. 1, in particular SEQ ID No. 2 for Cucumis melo, SEQ ID No. 3 for Citrullus lanatus, SEQ ID No. 4 for Cucurbita pepo, SEQ ID No. 5 for Solanum lycopersicum, SEQ ID No. 6 for Capsicum annuum (FIG. 1).

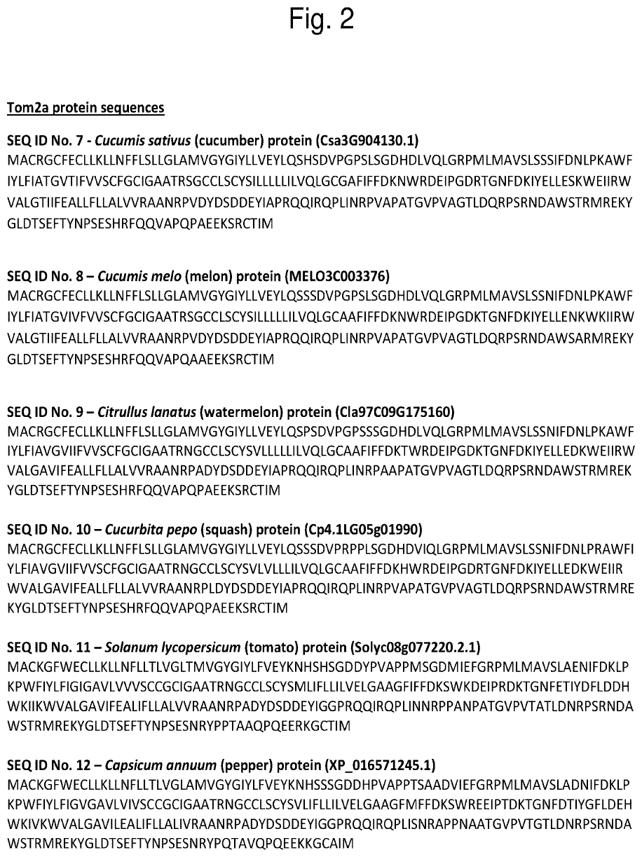

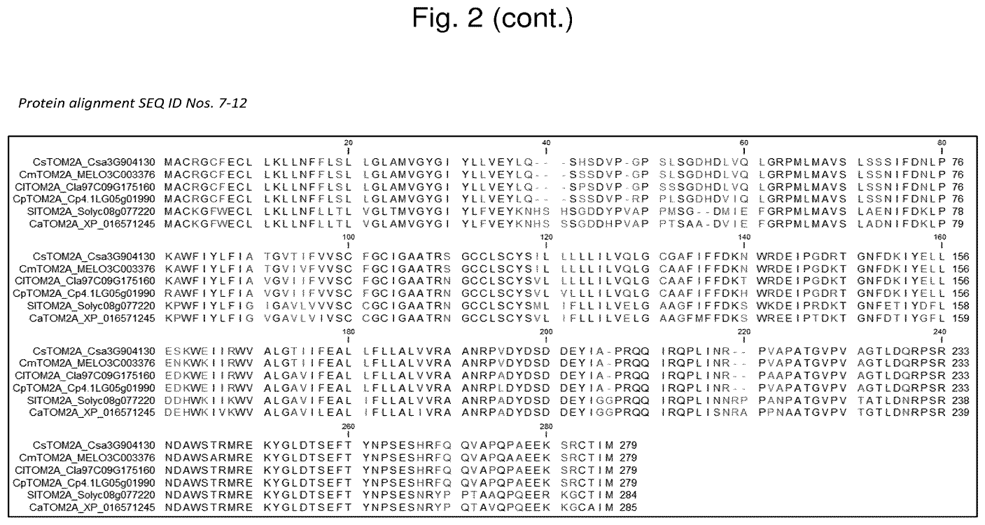

The wildtype Tom2a gene encodes a protein comprising SEQ ID No. 7 in Cucumis sativus, or encodes a Tom2a protein comprising a homologous sequence in another crop having at least 70% sequence similarity to SEQ ID No. 7, in particular SEQ ID No. 8 in Cucumis melo, SEQ ID No. 9 in Citrullus lanatus, SEQ ID No. 10 in Cucurbita pepo, SEQ ID No. 11 in Solanum lycopersicum, SEQ ID No. 12 in Capsicum annuum (FIG. 2).

The present invention relates to a modified Tom2a gene that has a modification that leads to a modified Tom2a protein. As used herein, a Tom2a protein is a protein comprising SEQ ID No. 7, or comprising a sequence having at least 70% sequence similarity to SEQ ID No. 7.

As used herein, a Tom2a gene is a gene encoding a Tom2a protein. As used herein, a Tom2a gene is a gene comprising a wildtype CDS sequence represented by SEQ ID No. 1 in Cucumis sativus, or a homologous gene in another crop comprising a sequence having at least 70% sequence identity to SEQ ID No. 1; or a gene encoding a Tom2a protein comprising SEQ ID No. 7 in Cucumis sativus; or a gene encoding a homologous Tom2a protein in another crop comprising a sequence having at least 70% sequence similarity to SEQ ID No. 7. As used herein, a gene optionally comprises the 5′-UTR sequence, the promoter, and the 3′-UTR sequence of that gene.

As used herein, a homologous Tom2a gene in another crop than Cucumis sativus comprises a homologous CDS sequence, which is a sequence having at least 70% sequence identity to SEQ ID No. 1, preferably at least 71%, 73%, 74%, 75%, 77%, 80%, 83%, 85%, 87%, 90%, 92%, 94%, 95%, 96%, 97%, 98%, 99%, or 100%. A homologous Tom2a protein in another crop than Cucumis sativus comprises a homologous protein sequence, which is a sequence having at least 70% sequence similarity to SEQ ID No. 7, preferably at least 72%, 75%, 77%, 80%, 81%, 83%, 85%, 87%, 90%, 93%, 95%, 96%, 97%, 98%, 99%, or 100%. A homologous Tom2a gene or protein is preferably a gene or protein present in a plant belonging to the Solanaceae family or in a plant belonging to the Cucurbitaceae family, in particular in Cucumis melo, Citrullus lanatus, Cucurbita pepo, Solanum lycopersicum, or Capsicum annuum.

As used herein, sequence identity or sequence similarity is the percentage of nucleotides or amino acids that is identical or similar between two sequences after proper alignment of those sequences. The person skilled in the art is aware of how to align sequences, for example by using a sequence alignment tool such as BLAST©, which can be used for both nucleotide sequences and protein sequences. To obtain the most significant result, the best possible alignment that gives the highest sequence identity or similarity score should be obtained. The percentage sequence identity or similarity is calculated through comparison over the length of the shortest sequence in the assessment, whereby in the present case a sequence that is included in such assessment represents a gene that at least comprises a start codon and a stop codon, or a complete protein encoded by such a gene. Sequence identity is used for comparison of nucleotide sequences. Sequence similarity is used to compare amino acid sequences, whereby conservative amino acid substitutions are deemed to be similar and is calculated herein based on the BLOSUM62 scoring matrix.

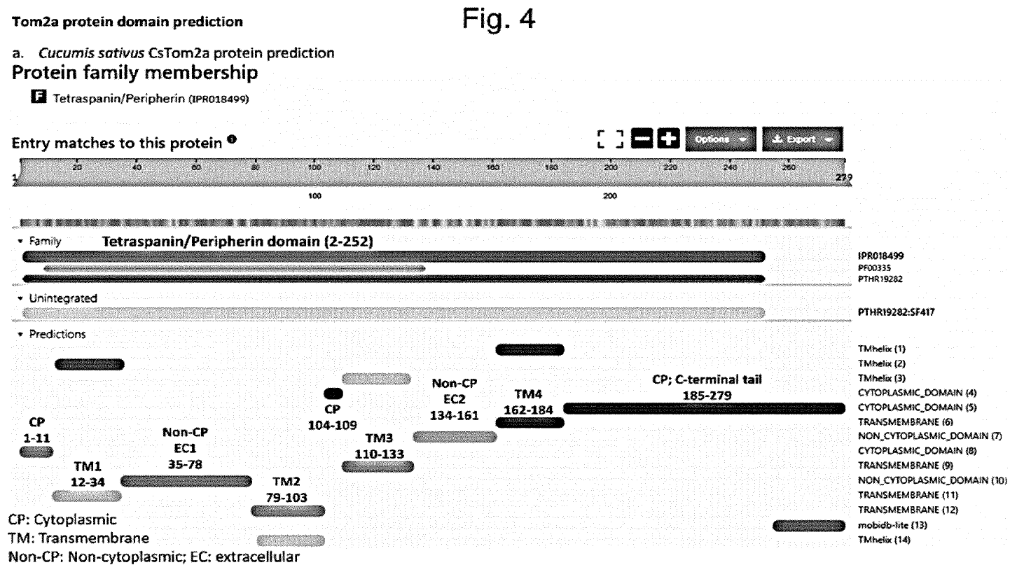

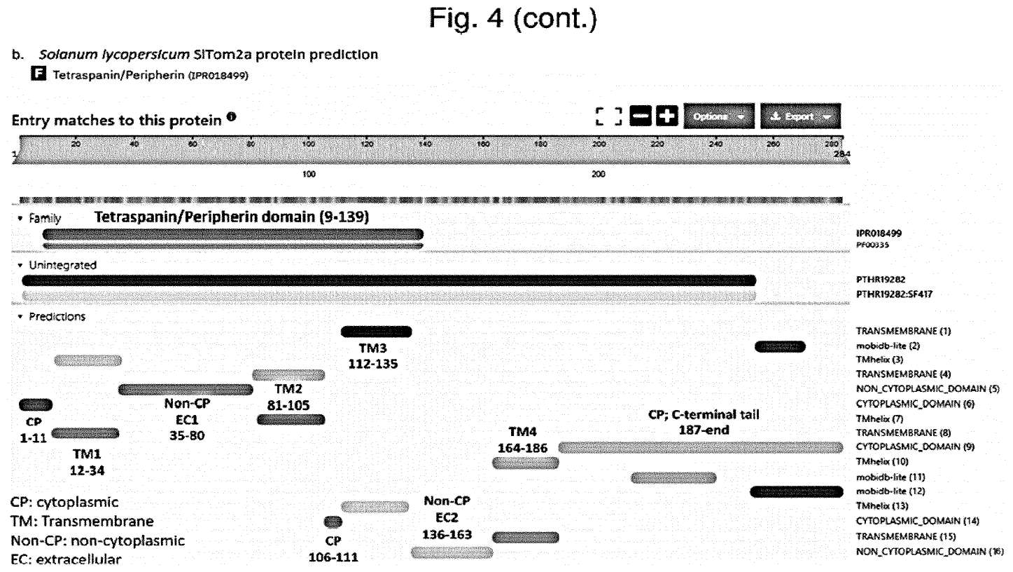

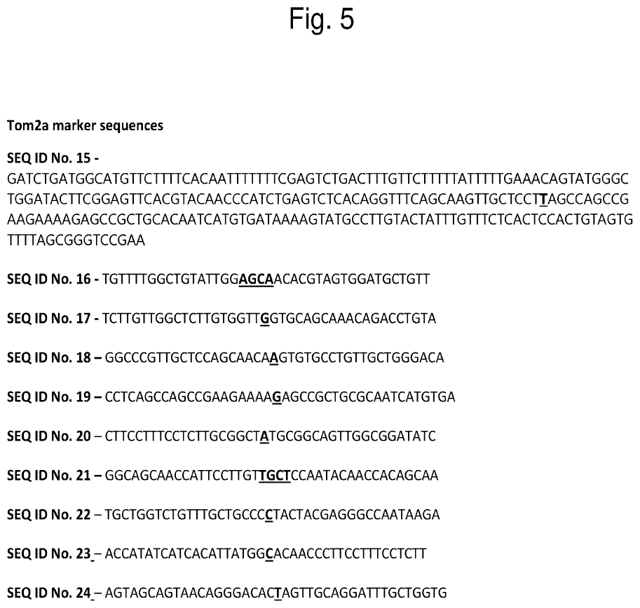

The Tom2a protein is a tetraspanin protein, which is a protein having a tetraspanin/peripherin domain. The protein comprises an N-terminal and a C-terminal tail, and four transmembrane domains (TM1-4) which are connected by two non-cytoplasmic loops and one very short cytoplasmic loop (FIG. 4). The second non-cytoplasmic, extracellular loop (EC2) harbours various conserved regions, and this loop is thought to play an essential role in tetraspanin function of both animal and plant cells. The polar residues of the transmembrane domains appear to be essential for stabilization of the tertiary protein structure. The C-terminal tail of the protein is also described as a crucial region for proper tetraspanin function.

In one embodiment, the modification in a Tom2a gene leads to a modified Tom2a protein that has a deletion, a substitution, or an insertion of at least one amino acid when compared to SEQ ID No. 7, SEQ ID No. 8, SEQ ID No. 9, SEQ ID No. 10, SEQ ID No. 11, or SEQ ID No. 12, or when compared to another homologous wildtype sequence of SEQ ID No. 7.

The modification in the Tom2a gene that leads to Tobamovirus resistance, in particular to resistance against CGMMV or ToBRFV, will change the activity and/or function of the encoded protein. The modification in the Tom2a gene of the invention comprises a modification resulting in an amino acid substitution in the encoded protein, a modification resulting in a premature stop codon, which results in truncation of the encoded protein, or a modification resulting in a frameshift. A modification resulting in a frameshift is a modification comprising an insertion or a deletion of at least one nucleotide. A frameshift mutation usually leads to a knockout of the gene, rendering it non-functional. Due to the modification, the encoded protein is absent, has a changed function, a reduced function, or it is non-functional. The modification of the present invention is in particular an induced modification. An induced modification is not naturally present, and leads to a non-natural modified Tom2a gene, and thereby to a non-natural modified Tom2a protein.

In one embodiment, the modified Tom2a protein has a modification in the second non-cytoplasmic, extracellular loop. In one embodiment, the modified Tom2a protein has a modification in a transmembrane domain, in particular in the TM2 domain. In one embodiment, the modified Tom2a protein has a modification in the cytoplasmic loop. In one embodiment the modified Tom2a protein has a modification in the C-terminal tail. A modification in the C-terminal tail is optionally resulting in a truncated protein, which leads to the absence or shortening of the C-terminal tail, which in that situation cannot perform its function. In one embodiment the modified Tom2a protein has a modification in the Non-CP EC1 loop or the Non-CP EC2 loop. In one embodiment, the modified Tom2a protein comprises a combination of said modifications.

In one embodiment the modified Tom2a protein is truncated and comprises only amino acids 1-267 of SEQ ID No. 7 or less, or the corresponding number of amino acids of a homologous Tom2a protein sequence, or the modified Tom2a protein comprises a modification in amino acids 268-279 of SEQ ID No. 7 that leads to a changed function, a reduced function, or a non-functional protein, or in the corresponding amino acids of a homologous Tom2a protein sequence, in particular of SEQ ID No. 8, SEQ ID No. 9, SEQ ID No. 10, SEQ ID No. 11, or SEQ ID No. 12.

In one embodiment the modified Tom2a gene is a knockout. In one embodiment the knockout leads to a protein that lacks the C-terminal tail; lacks the TM4 domain and the C-terminal tail; lacks the Non-CP EC2 loop and the TM4 domain and the C-terminal tail; lacks the TM3 domain and the Non-CP EC2 loop and the TM4 domain and the C-terminal tail; lacks the CP loop and the TM3 domain and the Non-CP EC2 loop and the TM4 domain and the C-terminal tail; or lacks the TM2 domain and the CP loop and the TM3 domain and the Non-CP EC2 loop and the TM4 domain and the C-terminal tail. In one embodiment the knockout leads to a protein that is truncated in the Non-CP EC1 loop. The knockout gene can be caused by a frameshift mutation, or by a mutation that leads to an early stop codon in another way, or by another mutation that leads to the loss-of-function of the gene or the encoded protein.

In one embodiment, a modification resulting in a modified Tom2a protein that increases Tobamovirus resistance comprises a C802T modification in SEQ ID No. 1 of cucumber, or a modification on a corresponding position in a homologous sequence of SEQ ID No. 1. Said nucleotide change results in a Q268stop modification in SEQ ID No. 7 of cucumber, or in a modification at the corresponding position of a homologous protein sequence of SEQ ID No. 7.

A modified Tom2a protein increasing Tobamovirus resistance comprises a protein having a Q268stop modification in SEQ ID No. 7 in cucumber, or a modification at the corresponding position of a homologous sequence having at least 70% sequence similarity to SEQ ID No. 7. This modification is a representative of a modification in the C-terminal tail of a Tom2a protein.

For Solanum lycopersicum, the Tom2a modifications GGC312-314del, A559G substitution, G673A, and A844G are described in co-pending application PCT/EP2021/070104. These modifications respectively lead to an A105del modification, an R187G substitution, a G225S substitution, and a T282A substitution in the S. lycopersicum Tom2a protein sequence of SEQ ID No. 11.

The present invention relates to extrapolated modifications of said S. lycopersicum modifications to the corresponding positions on other Tom2a genes, which lead to increased Tobamovirus resistance, in particular increased CGMMV or ToBRFV resistance. Extrapolation of amino acid positions can be done using a protein sequence alignment, which is presented for particular Tom2a proteins mentioned herein in FIG. 2.

In one embodiment, a modification resulting in a modified Tom2a protein that increases Tobamovirus resistance comprises one or more of a TGC306-308del modification, a C553G modification, a G658A modification, or a A829G modification in SEQ ID No. 1 of cucumber, or one or more modifications on corresponding positions in a homologous sequence of SEQ ID No. 1. Said nucleotide changes respectively result in a A103del modification, a R185G amino acid substitution, a G220S amino acid substitution, or a T277A amino acid substitution in SEQ ID No. 7 of cucumber, or in a modification at the corresponding position of a homologous protein sequence of SEQ ID No. 7.

A modified Tom2a protein increasing Tobamovirus resistance comprises a protein having an A103del modification, a R185G amino acid substitution, a G220S amino acid substitution, or a T277A amino acid substitution in SEQ ID No. 7 in cucumber, or a modification at the corresponding position of a homologous sequence having at least 70% sequence similarity to SEQ ID No. 7. An A103del modification is a representative of a modification in a transmembrane domain, in particular in the TM2 domain. This modification potentially also impacts the short cytoplasmic loop. The R185G, G220S, and T277A modifications are representatives of a modification in the C-terminal tail of a Tom2a protein (see FIG. 4).

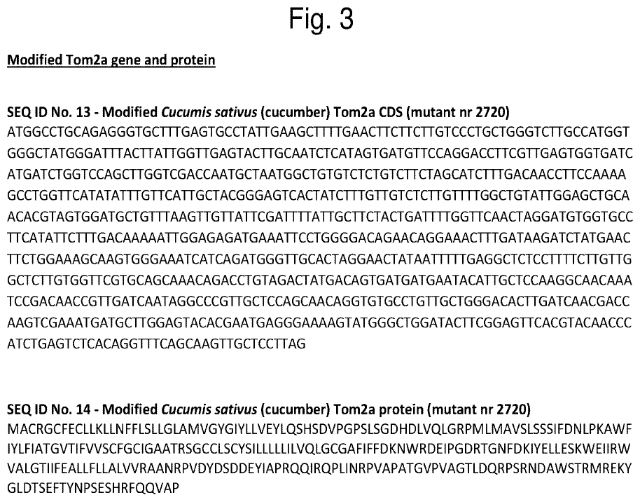

The CDS of a modified Tom2a gene in cucumber, resulting in a modified Tom2a protein, that increases Tobamovirus resistance, having a Q268stop modification is presented in SEQ ID No. 13, and the resulting truncated protein is presented in SEQ ID No. 14 (FIG. 3).

In one embodiment, the modified Tom2a gene encodes a modified Tom2a protein, comprising a combination of herein disclosed modifications on any of the corresponding positions in SEQ ID No. 7, SEQ ID No. 8, SEQ ID No. 9, SEQ ID No. 10, SEQ ID No. 11, or SEQ ID No. 12, or on another homologous sequence having at least 70% sequence similarity to SEQ ID No. 7. In one embodiment, the protein encoded by the modified Tom2a gene has retained a combination of two, three, or four of these modifications. Table 2 gives an overview of a number of said nucleotide and resulting amino acid modifications.

TABLE 2
Certain Tom2a modifications correlating
with Tobamovirus resistance

| Modifications based | | | | | |
|---|---|---|---|---|---|
| on SEQ ID No. 1 | | | | | |
| (CDS) and SEQ ID | | | | | |
| No. 7 (protein) | | | | | |
| positions | Tom2a | Tom2a | Tom2a | Tom2a | Tom2a |
| CDS SNP | C802T | TGC306- | C553G | G658A | A829G |
| modification | | 308del | | | |
| Susceptible wildtype | C | TGC | C | G | A |
| nucleotide (SEQ ID | | | | | |
| No. 1) | | | | | |
| Resistant nucleotide | T | deleted | G | A | G |
| Tom2a protein | Q268stop | A103del | R185G | G220S | T277A |
| modification | | | | | |

As used herein, at least 70% sequence identity relates to a sequence having in order of increased preference at least 70%, 71%, 73%, 74%, 75%, 77%, 80%, 81%, 83%, 85%, 87%, 90%, 92%, 94%, 95%, 96%, 97%, 98%, or 99% sequence identity.

As used herein, at least 70% sequence similarity relates to a sequence having in order of increased preference at least 70%, 72%, 75%, 77%, 80%, 81%, 83%, 85%, 87%, 90%, 93%, 95%, 96%, 97%, 98%, or 99% sequence similarity.

As used herein, an ‘X000Y’ mutation, modification, SNP, or substitution means that the wildtype sequence has a nucleotide or amino acid X on position 000, which is changed to nucleotide or amino acid Y in the modified sequence. The indication ‘X000del’ means that the modification is a deletion of the nucleotide or amino acid on that position or positions; the indication ‘X000stop’ means that the modification comprises the protein being truncated starting from that position, which is the result of the gene having a mutation leading to a premature stop codon.

The invention further relates to a seed that comprises a modified Tom2a gene of the invention, wherein a plant grown from said seed is a plant of the invention. In a preferred embodiment, the modified Tom2a gene of the invention is homozygously present in the seed. The invention also relates to seed produced by a plant of the invention, wherein the seed harbors the modified Tom2a gene of the invention, and as such, a plant grown from said seed is a plant of the invention. The invention also relates to use of said seed for the production of a plant of the invention, by growing said seed into a plant. The invention also relates to a plant part of a plant of the invention, which comprises a fruit, or a seed, wherein the plant part comprises a modified Tom2a gene in its genome.

The invention also relates to a fruit harvested from a plant of the invention, wherein the fruit comprises the modified Tom2a gene of the invention in its genome. This fruit is also referred to herein as ‘the fruit of the invention’. Moreover, the invention also relates to a food product or a processed food product comprising a fruit of a plant of the invention, or part of said fruit. The food product may have undergone one or more processing steps. Such a processing step might comprise, but is not limited to, any one of the following treatments or combinations thereof: peeling, cutting, washing, juicing, cooking, cooling or preparing a salad mixture comprising the fruit of the invention. The processed form that is obtained is also part of this invention

The invention further relates to a method for seed production, comprising growing a plant from a seed of the invention that comprises a modified Tom2a gene of the invention, allowing the plant to produce a fruit with seed, harvesting the fruit, and extracting those seed. Production of the seed is suitably done by crossing with itself or with another plant that is optionally also a plant of the invention. The seed that is so produced will grow into a plant that comprises the modified Tom2a gene of the invention. In a preferred embodiment the modified Tom2a gene is homozygously present in a plant used in seed production. The method in particular relates to production of a Cucumis sativus or a Solanum lycopersicum seed.

The invention further relates to hybrid seed and to a method for producing said hybrid seed, comprising crossing a first parent plant with a second parent plant and harvesting the resultant hybrid seed, wherein the first parent plant and/or the second parent plant is a plant of the invention comprising a modified Tom2a gene of the invention. The resulting hybrid seed, and the hybrid plant that can be grown from the hybrid seed, is also a part of the invention. In a preferred embodiment, both parent plants comprise the modified Tom2a gene of the invention homozygously, and the hybrid seed comprises the modified Tom2a gene of the invention homozygously. The hybrid seed is in particular seed of Cucumis sativus or of Solanum lycopersicum.

The present invention also relates to a method for producing a plant that has increased resistance to a virus of the genus Tobamovirus, in particular to CGMMV or to ToBRFV, comprising introducing a modification in a Tom2a gene, which modification leads to increased resistance. The introduced modification comprises a deletion, a substitution, or an insertion in the coding sequence of a Tom2a gene. Such modifications can for example lead to a codon change, a premature stop codon, or a frameshift. The introduced modification preferably leads to a modified Tom2a protein, which comprises an amino acid substitution, a truncated protein due to a premature stop codon, a deletion of one or more amino acids, or a changed amino acid sequence due to a frameshift. The introduction of such a modification can be done by a mutagenesis approach using a chemical compound, such as ethyl methane sulphonate (EMS); or by using physical means, such as UV-irradiation, fast neutron exposure, or other irradiation techniques.

Introduction of a modification can also be done using a more specific, targeted approach including targeted genome editing by means of homologous recombination, oligonucleotide-based mutation introduction, zinc-finger nucleases (ZFN), transcription activator-like effector nucleases (TALENs) or Clustered Regularly Interspaced Short Palindromic Repeat (CRISPR) systems, which comprises CRISPR/Cas systems. An endogenous susceptible Tom2a gene can thus be edited and modified into a Tom2a gene that confers increased Tobamovirus resistance.

The invention in particular relates to a method for producing a plant of the family Cucurbitaceae or Solanaceae that has increased Tobamovirus resistance, in particular increased CGMMV for the Cucurbitaceae or ToBRFV resistance for the Solanaceae, comprising introducing a modification in a Tom2a gene comprising SEQ ID No. 1, SEQ ID No. 2, SEQ ID No. 3, SEQ ID No. 4, SEQ ID No. 5, or SEQ ID No. 6, or in another homologous sequence having at least 70% sequence identity to SEQ ID No. 1. The method in particular comprises the introduction of a modification that leads to a Tom2a protein that has a changed function, a reduced function, or is non-functional. In one embodiment, the introduced modification leads to a truncated protein, wherein the truncated protein comprises the first 267 remaining amino acids of SEQ ID No. 7 or less, or the corresponding number of amino acids of a homologous Tom2a protein sequence, which in particular comprises SEQ ID No. 8, SEQ ID No. 9, SEQ ID No. 10, SEQ ID No. 11, or SEQ ID No. 12. In one embodiment, the introduced modification leads to a modification in one or more of the amino acids 268-279 of SEQ ID No. 7, which results in a changed function, a reduced function, or a non-functional protein, or in the corresponding amino acids of a homologous Tom2a protein sequence, in particular SEQ ID No. 8, SEQ ID No. 9, SEQ ID No. 10, SEQ ID No. 11, or SEQ ID No. 12. The method in particular relates to producing a Cucumis sativus plant having increased CGMMV resistance.

Introduction of a modified Tom2a gene of the invention can be done through introgression from a donor plant comprising said modified Tom2a gene, in particular from another plant that is resistant to a Tobamovirus and in which a modified Tom2a gene of the invention was identified, into a recipient plant that does not carry a modified Tom2a gene, or which carries a modified Tom2a gene heterozygously. Breeding methods such as crossing and selection, backcrossing, recombinant selection, or other breeding methods that result in the transfer of a genetic sequence from a donor plant to a recipient plant can be used. A donor plant, which is preferably a resistant plant, can be of the same species or of a different and/or wild species. Difficulties in crossing between species can be overcome through techniques known in the art such as embryo rescue, or cis-genesis can be applied. A plant produced by such method is also a part of the invention.

Alternatively, the modified Tom2a gene of the invention can be transferred or introduced from another, sexually incompatible, plant, for example by using a transgenic approach. Techniques that can suitably be used comprise general plant transformation techniques known to the skilled person, such as the use of an Agrobacterium-mediated transformation method.

The present invention relates to the use of a modified Tom2a gene for increasing Tobamovirus resistance in a plant. The invention also relates to the use of a modified Tom2a gene for the development of a plant of the Solanaceae or Cucurbitaceae that has increased resistance to a Tobamovirus, in particular to CGMMV or ToBRFV. The use of the modified Tom2a gene comprises introducing said gene into a plant.

The invention additionally relates to the use of a plant of the invention in plant breeding. The invention thus also relates to a breeding method for the development of a cultivated, preferably agronomically elite, plant that has increased resistance to a Tobamovirus, in particular to CGMMV or ToBRFV, wherein a plant comprising the modified Tom2a gene of the invention is used for conferring said resistance to another plant, by transferring the modified Tom2a gene.

The invention also relates to a method for the production of a plant which has increased resistance to a Tobamovirus, in particular to CGMMV or ToBRFV, said method comprising:
a) crossing a plant of the invention, which comprises a modified Tom2a gene of the invention, with another plant;
b) optionally performing one or more rounds of selfing and/or crossing of the plant resulting from the cross of step a) to obtain a further generation population;
c) selecting from the population resulting from the cross of step a), or from the further generation population of step b), a plant that comprises the modified Tom2a gene as defined herein, which plant has increased resistance to a Tobamovirus, in particular to CGMMV or ToBRFV.

In a preferred embodiment, above method relates to the production of a Cucumis sativus plant with increased CGMMV resistance or a Solanum lycopersicum plant with increased ToBRFV resistance.

The invention also relates to a method for the production of a plant which has increased resistance to a Tobamovirus, in particular to CGMMV or ToBRFV, said method comprising:
a) crossing a first parent plant comprising a modified Tom2a gene of the invention with a second parent plant, which is a plant not comprising a modified Tom2a gene of the invention;
b) backcrossing the plant resulting from step a) with the second parent plant for at least three generations;
c) selecting from the third or higher backcross population a plant that comprises at least the modified Tom2a gene of the first parent plant of step a).

In a preferred embodiment, the above method relates to the production of a Cucumis sativus plant with increased CGMMV resistance or a Solanum lycopersicum plant with increased ToBRFV resistance.

The invention additionally provides for a backcrossing method for introducing another desired trait into a plant that has increased resistance to a Tobamovirus, in particular to CGMMV or ToBRFV, comprising:
a) crossing a plant comprising a modified Tom2a gene of the invention with a second plant that comprises the other desired trait to produce F1 progeny;
b) optionally selecting in the F1 for a plant that comprises the modified Tom2a gene and the other desired trait;
c) crossing the optionally selected F1 progeny with either parent, to produce backcross progeny;
d) selecting backcross progeny comprising the increased Tobamovirus resistance and the other desired trait; and
e) optionally repeating steps c) and d) one or more times in succession to produce selected fourth or higher backcross progeny that comprises increased Tobamovirus resistance and the other desired trait.

Backcrossing is optionally done until the backcross progeny is stable and can be used as a parent line, which can be reached after 3 up to 10 backcrosses.

In one embodiment, the other desired trait is resistance to the same Tobamovirus for which resistance is increased through the presence of the modified Tom2a gene, but the resistance of the other desired trait is caused by a different gene then the Tom2a gene. This approach, known as stacking of resistances, leads to a stronger and more durable Tobamovirus resistance in a plant of the invention, in particular to stronger and more durable CGMMV or ToBRFV resistance.

In a preferred embodiment, the above backcrossing method relates to the introduction of another desired trait in a Cucumis sativus plant that has increased CGMMV resistance or in a Solanum lycopersicum plant that has increased ToBRFV resistance.

Optionally, selfing steps are performed after any of the crossing or backcrossing steps in above described methods. Selection of a plant comprising the modified Tom2a gene of the invention and the other desired trait can alternatively be done following any crossing or selfing step of the method. The other desired trait can be selected from, but is not limited to, the following group: resistance to bacterial, fungal or viral diseases, insect or pest resistance, improved germination, plant size, plant type, plant vigor, improved shelf-life, larger fruit size, smaller fruit size, improved fruit quality, parthenocarpic fruit set, water stress tolerance, heat stress tolerance, cold stress tolerance, and male sterility. The invention includes a plant produced by this method and a fruit obtained therefrom.

The invention further relates to a method for the production of a plant comprising a modified Tom2a gene of the invention, by using tissue culture or by using vegetative propagation.

The invention further provides a method for the production of a plant comprising the modified Tom2a gene of the invention by using a doubled haploid generation technique to generate a doubled haploid line that is completely homozygous, and therefore homozygously comprises the modified Tom2a gene of the invention, and has increased resistance to a Tobamovirus, in particular to CGMMV or ToBRFV.

The invention further relates to a method for the production of a plant comprising the modified Tom2a gene of the invention, wherein the presence of said modified Tom2a gene leads to increased Tobamovirus resistance, which method comprises growing a seed comprising said modified Tom2a gene into the said plant.

The present invention relates to a method for identification of a plant comprising a modified Tom2a gene of the invention, wherein the identification comprises determining the presence of a modification in a Tom2a gene comprising SEQ ID No. 1, SEQ ID No. 2, SEQ ID No. 3, SEQ ID No. 4, SEQ ID No. 5, SEQ ID No. 6, or in a homologous sequence of SEQ ID No. 1, and optionally analyzing if the plant comprising the modification has increased resistance to a Tobamovirus, in particular to CGMMV or ToBRFV. Said method relates to the identification of a plant of a species belonging to the Cucurbitaceae or Solanaceae, and in particular to the identification of a plant of a species belonging to the genus Cucumis or the genus Solanum. The method preferably relates to the identification of a Cucumis sativus plant comprising a modified Tom2a gene, or of a wild tomato species, in particular of the species Solanum pimpinellifolium comprising a modified Tom2a gene, or to the identification of a plant of the species Solanum lycopersicum comprising a modified Tom2a gene.

Determining the presence of a modification in a Tom2a gene comprises identification of any modification in SEQ ID No. 1, SEQ ID No. 2, SEQ ID No. 3, SEQ ID No. 4, SEQ ID No. 5, SEQ ID No. 6, or in another homologous sequence of SEQ ID No. 1, that leads to increased Tobamovirus resistance, in particular increased CGMMV or ToBRFV resistance. Determining the presence of a modification includes any of the modifications as described herein, in particular the modifications C802T, TGC306-308del, C553G, G658A, or A829G in SEQ ID No. 1, as presented in Table 2. Determining the presence of any modification can be done through sequence comparison between the wildtype Tom2a sequence and the Tom2a sequence of a plant to be analysed, whereby methods for performing sequence comparison are known to the skilled person. Determining the presence of a specific modification is suitably done by using a marker that is designed to identify such modification, as its sequence comprises that particular modification in relation to the wildtype sequence.

The present invention further relates to a method for selection of a plant, which has increased resistance to a Tobamovirus, in particular to CGMMV or ToBRFV, the method comprising identification of a modified Tom2a gene of the invention in a plant, and subsequently selecting said plant as a plant which has increased resistance to a Tobamovirus, in particular to CGMMV or ToBRFV. Optionally, the increased virus resistance can be confirmed by performing a bio-assay as described herein. The selected plant obtained by such method is also a part of this invention.

The invention also relates to a method of testing a plant for the presence of the modified Tom2a gene of the invention that increases Tobamovirus resistance, in particular CGMMV or ToBRFV resistance, comprising detecting the presence of a polymorphism that leads to said increased resistance in SEQ ID No. 1, SEQ ID No. 2, SEQ ID No. 3, SEQ ID No. 4, SEQ ID No. 5, SEQ ID No. 6, or in another homologous sequence of SEQ ID No. 1, in the genome of the plant. The method of testing a plant for the presence of the modified Tom2a gene of the invention optionally further comprises selecting a plant that comprises said modified Tom2a gene as a plant having increased Tobamovirus resistance, in particular increased CGMMV or ToBRFV resistance. The plant that is thus selected can subsequently be used as a source for introgressing the modified Tom2a gene into a plant lacking said allele.

The invention also relates to propagation material suitable for producing a plant of the invention, wherein the propagation material is suitable for sexual reproduction, and is in particular selected from a microspore, pollen, an ovary, an ovule, an embryo sac, and an egg cell; or is suitable for vegetative reproduction, and is in particular selected from a cutting, a root, a stem cell, and a protoplast; or is suitable for tissue culture of regenerable cells, and is in particular selected from a leaf, pollen, an embryo, a cotyledon, a hypocotyl, a meristematic cell, a root, a root tip, an anther, a flower, a seed, and a stem; wherein the plant produced from the propagation material comprises the modified Tom2a gene of the invention as defined herein that provides increased resistance to a Tobamovirus, in particular to CGMMV or ToBRFV. A plant of the invention may be used as a source of the propagation material.

The invention further relates to a cell comprising the modified Tom2a gene of the invention as defined herein. A cell of the invention can be obtained from, or be present in, a plant of the invention. Such a cell may either be in isolated form, or a part of the complete plant, or from a part thereof, and still constitutes a cell of the invention because such a cell comprises the genetic information that determines the modified Tom2a gene as described herein. Each cell of a plant of the invention carries the modified Tom2a gene of the invention, and thereby the genetic information that leads to increased Tobamovirus resistance. A cell of the invention may also be a regenerable cell that can regenerate into a new plant of the invention. The presence of the genetic information in this context is the presence of the modified Tom2a gene of the invention, wherein the modified Tom2a gene is as defined herein.

The invention further relates to plant tissue of a plant of the invention, which comprises the modified Tom2a gene of the invention. The tissue can be undifferentiated tissue or already differentiated tissue. Undifferentiated tissue is for example a stem tip, an anther, a petal, or pollen, and can be used in micropropagation to obtain new plantlets that are grown into new plants of the invention. The tissue can also be grown from a cell of the invention.

The invention moreover relates to progeny of a plant, a cell, a tissue, or a seed of the invention, which progeny comprises the modified Tom2a gene of the invention. Such progeny can in itself be a plant, a cutting, a cell, a tissue, or a seed.

As used herein, ‘progeny’ is intended to mean the first and all further descendants, such as an F1, F2, or further generation, from a cross with a plant of the invention, wherein a cross comprises a cross with itself or a cross with another plant, and wherein a descendant that is determined to be progeny comprises the modified Tom2a gene of the invention that leads to increased Tobamovirus resistance. Progeny also encompasses a plant or plant material that comprises the modified Tom2a gene of the invention, and that is obtained from a plant, or progeny of a plant, of the invention by vegetative propagation or another form of multiplication.

The invention further relates to a part of a plant of the invention that is suitable for sexual reproduction, which plant part comprises the modified Tom2a gene of the invention as defined herein. Such a part is for example selected from the group consisting of a microspore, a pollen, an ovary, an ovule, an embryo sac, and an egg cell.

Additionally, the invention relates to a part of a plant of the invention that is suitable for vegetative reproduction, which is in particular a cutting, a root, a stem, a cell, or a protoplast that comprises the modified Tom2a gene of the invention as defined herein. A part of a plant as previously mentioned is considered propagation material. The plant that is produced from the propagation material comprises the modified Tom2a gene of the invention, the presence of which leads to increased Tobamovirus resistance.

The invention further relates to tissue culture of a plant of the invention, which is also propagation material, and which comprises the modified Tom2a gene of the invention in its genome. The tissue culture comprises regenerable cells. Such tissue culture can be selected or derived from any part of the plant, in particular from a leaf, pollen, an embryo, a cotyledon, a hypocotyl, a meristematic cell, a root, a root tip, an anther, a flower, a seed, or a stem. The tissue culture can be regenerated into a plant comprising the modified Tom2a gene of the invention, wherein the regenerated plant has increased Tobamovirus resistance and is also part of the invention.

The invention also relates to a marker for the identification of a modified Tom2a gene in a plant, which marker comprises any of the modifications in a Tom2a gene as described herein and can thereby identify said modifications. Such marker for identification comprises a nucleotide sequence that includes a particular polymorphism in its sequence, when compared to the same sequence stretch in a wildtype Tom2a gene, which polymorphism leads to a modification in the encoded protein that changes the function or activity of the Tom2a protein.

A marker of the invention is in particular a marker comprising in its sequence any one of the following: a T at position 802, a deletion at positions 306-308, a G at position 553, an A at position 658, and a G at position 829, wherein the positions are relative to SEQ ID No. 1, and wherein the nucleotide is representative of a C802T, TGC306-308del, C553G, G658A, or A829G modification in SEQ ID No. 1, or comprises in its sequence nucleotides representative of a polymorphism that leads to a modification on a corresponding position in a homologous sequence thereof, and is thereby suitable for identifying any of those modifications in a Tom2a gene.

Nucleotide sequences comprising said polymorphisms, that are thereby suitable to identify said polymorphisms in SEQ ID No. 1, are presented as SEQ ID No. 15 for identifying a C802T modification, SEQ ID No. 16 for identifying a TGC306-308del modification, SEQ ID No. 17 for identifying a C553G modification, SEQ ID No. 18 for identifying a G658A modification, and SEQ ID No. 19 for identifying a A829G modification. Nucleotide sequences comprising said polymorphisms, that are suitable to identify corresponding polymorphisms on the corresponding positions in SEQ ID No. 5, are presented as SEQ ID No. 20 for identifying a C817T modification, SEQ ID No. 21 for identifying a GGC312-314del modification, SEQ ID No. 22 for identifying an A559G modification, SEQ ID No. 23 for identifying a G673A modification, and SEQ ID No. 24 for identifying an A844G modification. Because the Tom2a gene is present on the minus-strand in the public S. lycopersicum genome, the marker sequences of SEQ ID No. 20-24 are presented as reversed complement sequences in relation to SEQ ID No. 5.

Optionally, the sequence to be used as a marker can be longer on either side of the modification, to ensure the sequence is unique in the genome and locates to the Tom2a gene. The sequences surrounding the polymorphisms are according to SEQ ID No. 1 for Cucumis sativus, according to SEQ ID No. 5 for Solanum lycopersicum or, for other crops, according to a sequence that has at least 70% sequence identity therewith, in particular SEQ ID No. 2, SEQ ID No. 3, SEQ ID NO. 4, or SEQ ID No. 6.

The invention also relates to the use of a marker for identification of a modified Tom2a gene, particularly a marker represented by any one of SEQ ID Nos. 15-24. The invention further relates to the use of a marker, in particular a marker as described herein, for identification of a modified Tom2a gene that leads to increased Tobamovirus resistance, in particular in a Cucumis sativus or a Solanum lycopersicum or a Solanum pimpinellifolium plant, and/or for selection of a Cucumis sativus or Solanum lycopersicum or Solanum pimpinellifolium plant comprising a modified Tom2a gene that leads to increased Tobamovirus resistance, in particular increased CGMMV or ToBRFV resistance. The invention also relates to selection of a plant identified by a marker as described herein and to the plant thus selected.

The present invention will be further illustrated in the Examples that follow and that are for illustration purposes only. The Examples are not intended to limit the invention in any way. In the Examples and in the application, reference is made to the following figures.

### EXAMPLES

### Example 1

Tom2a TILLING Mutants in Cucumis sativus

To develop new variation for various traits, a large TILLING population was developed in Cucumis sativus, using ethyl methane sulphonate (EMS) as the mutagenic agent. In the search for new resistances, a TILLING approach was applied for various genes, and multiple screens were performed in more than 3000 plants over the years. In a screen performed in 2016, three mutants in the Tom2a gene were identified; in 2017 two more were found, and in 2019 a sixth mutant was identified. All of these mutants were selected for follow-up observation, since they resulted in potentially interesting amino acid changes or premature stop codons (Table 3).

To obtain plants that had the mutation of interest, while other mutations resulting from the EMS treatment would be removed, first several rounds of selfing were performed that focused on maintaining the specifically identified mutations in the Tom2a gene.

TABLE 3
Tom2a TILLING mutants in C. sativus

| | Tom2a TILLING modifications | | | | | |
|---|---|---|---|---|---|---|
| | obtained in C. sativus | | | | | |
| | Tom2a | Tom2a | Tom2a | Tom2a | Tom2a | Tom2a |
| CDS Tom2a SNP | G11A | G64A | G411A | G751A | C802T | G824A |
| modification | | | | | | |
| Tom2a protein | R4K | G22S | W137stop | E251K | Q268stop | R275H |
| modification | | | | | | |

The screen from 2016 included the very interesting C802T mutant, which resulted in a truncated protein that only comprised the first 267 amino acids of the wildtype protein. This mutant, which was assigned the code ‘mutant 2720’, was among the first to have gone through several selfing steps, and was selected for a first phenotyping observation round for virus resistance. In addition, backcrosses with the line used for developing the EMS population was done, designated ‘KK1’, and segregating populations were developed. In a segregating population, good phenotypic comparison of plants with and without the specific mutation can be done.

### Example 2

Phenotyping of Q268stop Mutant for CGMMV Resistance in Cucumis sativus

A population of cucumber plants of Q268stop mutant ‘2720’ was selfed two times, while selecting for the presence of the C802T mutation. After these rounds of selfing, a phenotyping assay for CGMMV resistance was performed.

To do the bio-assay, seeds of the mutant plants were sown, and the CGMMV susceptible KK1 line and susceptible variety Ventura F1 were included as controls. Sowing was done in a standard seedling tray at 23° C. After 5 days, 30 seedlings of the mutant and 30 seedlings of the controls were transplanted to a larger tray. The plants of the mutant line and the controls were divided over three replicates. Unfortunately, only 17 plants of the mutant line survived; 7 in the first replicate, 4 in the second, and 6 in the third replicate. One week after sowing, the transplants were inoculated and transferred to a temperature regime of 20° C. by day and 18° C. by night. Inoculum was prepared by grounding leaves of cucumber plants that were infected with CGMMV in a 0.01 M phosphate buffer (pH 7.0). The seedlings were then dusted with carborundum powder prior to gently rubbing the leaf with inoculum.

Resistance was scored on a scale of 1-5, according to the description of the scales of the scores in Table 1. Observation of the symptoms on the young cucumber plants in the bio-assay was done 14-21 days after inoculation (dai). Scores are presented in Table 4; a/b/c/d/e corresponds to a score in scale 1/2/3/4/5. The mutant showed a clearly increased CGMMV resistance over the susceptible controls.

TABLE 4
phenotyping of the Q268stop mutant for CGMMV resistance

| | Total score | | | | Average |
|---|---|---|---|---|---|
| CGMMV | over all | | | | over all |
| phenotyping | replicates | Score rep 1 | Score rep 2 | Score rep 3 | replicates |
| Ventura | 0/0/0/0/30 | 0/0/0/0/10 | 0/0/0/0/10 | 0/0/0/0/10 | 5.0 |
| KK1 | 0/0/1/9/20 | 0/0/0/0/10 | 0/0/1/9/0 | 0/0/0/0/10 | 4.6 |
| Q268stop mutant | 0/11/0/6/0 | 0/7/0/0/0 | 0/4/0/0/0 | 0/0/0/6/0 | 2.7 |

### Example 3

### Modification of a Tom2a Gene to Obtain Resistance to a Tobamovirus

Modifications are introduced in seed of a plant of interest in which resistance to a Tobamovirus is needed, in particular to CGMMV in a plant of the Cucurbitaceae, or to ToBRFV in a plant of the Solanaceae. The modification is introduced through mutagenesis, such as an EMS treatment, through radiation means, or through a specific targeted approach, such as a CRISPR/Cas system. When a non-targeted approach such as EMS is used, this is combined with an identification technique such as TILLING. In this way, both for mutagenesis as well as a targeted modification means, a modification in a Tom2a gene can be generated and identified. The skilled person is familiar with these means for introducing modifications into the genome of a plant of interest.

Modified seed is then germinated and plants are grown, which are crossed or selfed to generate M2 or further generation seed. Subsequently a plant screen is performed to identify the modifications in a Tom2a gene, based on comparison to the wildtype sequence of the Tom2a gene of that species. For Cucumis sativus for example, comparison to SEQ ID No. 1 should be done; for Solanum lycopersicum, comparison to SEQ ID No. 5 should be done. The skilled person is familiar with TILLING to identify mutations in specific genes (McCallum et. al. (2000) Nature Biotechnology, 18: 455-457), and with techniques for identifying nucleotide changes such as DNA sequencing, amongst others.

Plants with a modified Tom2a gene are homozygous or made homozygous by selfing, crossing, or the use of doubled haploid techniques which are familiar to the skilled person. Plants identified and selected on the basis of a modification in a Tom2a gene can then be tested for resistance to a Tobamovirus, in particular for resistance to CGMMV or ToBRFV. A plant that is produced, identified and selected in this way is confirmed to have their virus resistance as a result from one or more modifications in the Tom2a gene.

### Example 4

Tom2a TILLING Mutants in Solanum lycopersicum

To develop new variation for various traits, a large TILLING population was developed in Solanum lycopersicum, using ethyl methane sulphonate (EMS) as the mutagenic agent. In the search for new Tobamovirus resistance, a TILLING approach was applied for various genes, and multiple screens were performed over the years. In the Tom2a gene, a total of ten mutants that led to protein changes were found. According to a SIFT prediction, four of those mutants were predicted to be tolerated, and the others were either deleterious or resulted in a truncated protein. These mutants, in particular the ones that are predicted to be deleterious or resulted in a truncated protein, were selected for follow-up phenotype observation, since they resulted in potentially interesting amino acid changes or premature stop codons (Table 5).

To obtain plants with only the mutation of interest, several rounds of selfing and backcrossing are being performed to get the suitable generation for doing a bio-assay on Tobamovirus resistance, in particular on ToBRFV resistance.

TABLE 5
Tom2a TILLING mutants in S. lycopersicum

| Tom2a TILLING | |
|---|---|
| mutants obtained | Result SIFT |
| in S. lycopersicum | prediction |
| W7stop | |
| R191K | TOLERATED |
| A172V | TOLERATED |
| A91T | TOLERATED |
| A2V | DELETERIOUS |
| P144L | DELETERIOUS |
| P227L | DELETERIOUS |
| C3Y | DELETERIOUS |
| P80L | DELETERIOUS |
| D45Y | TOLERATED |

### Example 5

Generation of Tom2a CRISPR Mutants in Solanum lycopersicum

An internal ToBRFV susceptible S. lycopersicum breeding line was used for developing targeted knockouts of the Tom2a gene, using a CRISPR/Cas9 editing system, to observe the effect on ToBRFV resistance. The susceptible S. lycopersicum line was determined to have the wildtype Tom2a sequence, represented by SEQ ID No. 5. The annotated gene sequence was used to design a large number of possible single guide RNAs (sgRNAs). Subsequently two sgRNAs were chosen that had a high on-target score and a low off-target score. The Tom2a gene comprises 6 exons; one of the sgRNAs targeted a location in exon 1. The other sgRNA targeted a location in exon 5. The used sgRNAs are presented in Table 6.

TABLE 6
sgRNA sequences

| sgRNA | SEQ ID | #RGEN Target (5′ to 3′) | Gene |
|---|---|---|---|
| nr | No. | sgRNA-PAM | name |
| 1 | SEQ ID | TGCGTGGAGTACACGTATGA | Tom2a |
| | No. 25 | | |
| 2 | SEQ ID | CCAGTTGCACCACCTATGAG | Tom2a |
| | No. 26 | | |

The two selected sgRNAs were used in Agrobacterium tumefaciens-mediated transformation with the CRISPR/Cas9 construct, according to the protocol as described in Pan, C. et al, CRISPR/Cas9-mediated efficient and heritable targeted mutagenesis in tomato plants in the first and later generations. Sci. Rep. 6, 24765 (2016).

The use of the combination of the two sgRNAs resulted in multiplex events in the Tom2a gene. Mutant plants were obtained that were affected in both targeted locations. Two plants with mutations in the Tom2a gene were selected. Both mutants comprised a 1 bp insertion in exon 1, at location 157 of SEQ ID No. 5, which led to a frameshift. One of the mutants comprised another event, a 1 bp insertion in exon 5, at location 737 of SEQ ID No. 5. The other mutant comprised a different second event, namely a 2 bp deletion in exon 5, of positions 736 and 737 of SEQ ID No. 5. The targeted mutations led to knockouts of the Tom2a gene. Seeds of the mutant plants were increased to the T2 generation. The mutations were homozygously present in the T2 generation, which generation was tested for ToBRFV resistance. Eight T2 lines having a combination of a mutation in exon 1 and exon 5 were selected for phenotyping.

### Example 6

Phenotyping for Symptoms and Virus Titer of Tom2a Knockouts in S. lycopersicum.

Eight T2 lines obtained from plants having homozygous knockouts of the Tom2a gene, that were obtained by the CRISPR/Cas9 editing method described in Example 5, were phenotyped in a ToBRFV bio-assay. All plants had the described 1 bp insertion in exon 1; lines 3.71, 3.64, and 3.32 also had the described 1 bp insertion in exon 5. Lines 3.62, 3.54, 3.38, 3.28, and 3.14 had next to the exon 1 insertion also the described 2 bp deletion in exon 5.

From all numbers, 20 seeds were sown in standard seedling trays and seedlings were transplanted to larger pots after 2-3 weeks. The transplanted seedlings were inoculated 4 weeks after sowing. As a susceptible control, the variety Eclipse F1 was included. From some lines, not all seeds were germinated. From lines 3.14 and 3.71, only 11 seedlings could be inoculated; from line 3.38, 10 seedlings were inoculated; and from line 3.64, only 6 seedlings were inoculated. Inoculum was prepared by grounding leaves of tomato plants that were infected with ToBRFV in a 0.01 M phosphate buffer (pH 7.0) mixed with celite. The seedlings were then dusted with carborundum powder prior to gently rubbing the leaf with inoculum. Resistance was scored on a scale of 0-5; the description of the scales of the scores can be found in Table 7. Observation of the symptoms on the young tomato plants in the bio-assay was done 14-21 days after inoculation (dai).

TABLE 7
scales ToBRFV resistance scores

| Score | Symptoms |
|---|---|
| 0 | No symptoms |
| 1 | Not clean, a single spot, some minor discoloration |
| 2 | Mosaic, clear visible symptoms |
| 3 | Severe mosaic, starting deformation in the head |
| 4 | Severe mosaic, necrosis on the stem, serious deformation in the |
| | head, spots in blisters |
| 5 | Dead plant |

All plants of the susceptible control nicely showed a score 4 during phenotyping, which indicated a well-performed experiment. The Tom2a knockout mutants that were included in the assay showed an extremely good level of resistance. Average scores were all 1.0 or lower; for line 3.64, which scored 1.0, only a limited number of plants could be included. Scores of the phenotypes are presented in FIG. 6.

Apart from the visual symptoms, also the measurement of the virus titer is a very relevant aspect of virus resistance. Virus titer was determined in ToBRFV infected leaf samples from 20 plants of each T2 line that was obtained as described in Example 5. As a susceptible control, the variety Eclipse F1 was used. From each plant a leaf punch of 6 mm in diameter was taken, and subsequently ground in 500 μl of PBS buffer solution. 50 μl of the resulting suspension was used in a 96-well KingFisher Flex isolation protocol, whereby isolation of the leaf material was done using the innuPREP DNA/RNA virus PLUS Kit. The samples were then analysed in a 96CFX qPCR thermocycler (Biorad) to get a Cq_ToBRFV value, which represents the number of cycles needed to obtain the virus PCR product, using a programme of 5 minutes on 50° C. and 20 sec. on 95° C., followed by 40 cycles of 10 sec. on 95° C. and 60 sec. on 60° C.

To be able to compare the values of samples of different sizes and backgrounds, the S. lycopersicum PHD reference gene, a tomato housekeeping gene, was included in the qPCR assay, which corrects any variation in the amount of sample material, and then yields a Cq_PHD value. To accurately determine the final value for virus titer the Delta Cq method was used, with PHD as a housekeeping gene and ToBRFV as the gene of interest. The final value is the Cq_corr, which is calculated as Cq_ToBRFV−Cq_PHD. A plant is determined to have a reduction of ToBRFV virus replication when the average Cq_corr was higher than −11.00, or when the average Cq-corr was at least 5.00 higher than the average Cq_corr of the susceptible control.

Virus titer results of all knockout mutants were very convincing. The average Cq_corr value of the susceptible control was −16.21. All mutants had an average score that was higher than −5.00, and most of them even had an average score higher than 0.00. Results of virus titer measurements are presented in FIG. 7.

It was concluded that Tom2a knockout mutants in S. lycopersicum result in a very high level of ToBRFV resistance, for both visual symptoms as well as virus titer in the plants.

The invention is further described by the following numbered paragraphs:
1. A plant comprising a modified Tom2a gene, the wildtype of which comprises SEQ ID No. 1, SEQ ID No. 2, SEQ ID No. 3, SEQ ID No. 4, SEQ ID No. 5, or SEQ ID No. 6, or comprises another homologous sequence having at least 70% sequence identity to SEQ ID No. 1, which modified gene leads to increased resistance to a Tobamovirus.
2. The plant of paragraph 1, which comprises a modification in the Tom2a gene that leads to a modified Tom2a protein that has a deletion, a substitution, or an insertion of at least one amino acid when compared to SEQ ID No. 7, SEQ ID No. 8, SEQ ID No. 9, SEQ ID No. 10, SEQ ID No. 11, or SEQ ID No. 12, or compared to another homologous sequence having at least 70% sequence similarity to SEQ ID No. 7.
3. The plant of paragraphs 1 or 2, wherein the modification results in a premature stop codon which leads to a truncated protein.
4. The plant of any one of paragraphs 1-3, wherein the Tom2a protein encoded by the modified Tom2a gene is absent, has a changed function, a reduced function, or is non-functional.
5. The plant of any one of paragraphs 1-4, wherein the modification in the Tom2a protein is present in a transmembrane domain or in the C-terminal tail.
6. The plant of any one of paragraphs 1-4, wherein the modification in the Tom2a protein is present in Non-CP EC1 or Non-CP EC2.
7. The plant of any one of paragraphs 1-6, wherein the modified Tom2a protein is truncated and comprises only amino acids 1-267 of SEQ ID No. 7 or less, or the corresponding number of amino acids of a homologous Tom2a protein sequence, or the modified Tom2a protein comprises a modification in amino acids 268-279 of SEQ ID No. 7 that leads to a changed function, a reduced function, or a non-functional protein, or in the corresponding amino acids of a homologous Tom2a protein sequence, in particular of SEQ ID No. 8, SEQ ID No. 9, SEQ ID No. 10, SEQ ID No. 11, or SEQ ID No. 12.
8. The plant of any one of paragraphs 1-7, wherein the modified Tom2a protein comprises SEQ ID No. 14.
9. The plant of any one of paragraphs 1-8, which is a plant of the family Cucurbitaceae or Solanaceae.
10. The plant of paragraph 9, which is a plant of the species Cucumis sativus, Cucumis melo, Citrullus lanatus, Cucurbita pepo, Solanum lycopersicum, or Capsicum annuum, preferably a plant of the species Cucumis sativus or Solanum lycopersicum.
11. The plant of paragraph 9 or 10, which is a plant of the family Cucurbitaceae and has increased resistance to CGMMV.
12. The plant of paragraph 9 or 10, which is a plant of the family Solanacae and has increased resistance to ToBRFV.
13. A modified Tom2a gene encoding a Tom2a protein comprising a deletion, a substitution, or an insertion of at least one amino acid when compared to SEQ ID No. 7, SEQ ID No. 8, SEQ ID No. 9, SEQ ID No. 10, SEQ ID No. 11, or SEQ ID No. 12, or compared to another homologous sequence having at least 70% sequence similarity to SEQ ID No. 7.
14. The modified Tom2a gene of paragraph 13, which modified gene comprises a premature stop codon and which encodes a truncated Tom2a protein
15. The modified Tom2a gene of paragraph 13 or 14, wherein the Tom2a protein encoded by the modified Tom2a gene is absent, has a changed function, a reduced function, or is non-functional.
16. The modified Tom2a gene of any one of paragraphs 13-15, wherein the encoded modified Tom2a protein is truncated and comprises only amino acids 1-267 of SEQ ID No. 7 or less, or the corresponding number of amino acids of a homologous Tom2a protein sequence, or the modified Tom2a protein comprises a modification in amino acids 268-279 of SEQ ID No. 7 that leads to a changed function, a reduced function, or a non-functional protein, or in the corresponding amino acids of a homologous Tom2a protein sequence, in particular of SEQ ID No. 8, SEQ ID No. 9, SEQ ID No. 10, SEQ ID No. 11, or SEQ ID No. 12.
17. The modified Tom2a gene of any one of paragraphs 13-16, wherein the presence of the modified gene in a plant leads to increased Tobamovirus resistance, in particular to increased CGMMV resistance in a plant of the family Cucurbitaceae, or to increased ToBRFV resistance in a plant of the family Solanaceae.
18. Seed, comprising a modified Tom2a gene as defined in any one of paragraphs 13-17, wherein the plant grown from the seed is a plant of any one of paragraphs 1-12.
19. Use of a marker for identification of a modified Tom2a gene that leads to increased Tobamovirus resistance, in particular a marker comprising any one of SEQ ID Nos. 15-24.
20. Use of the marker of paragraph 19 for identification of a plant having a modified Tom2a gene.
21. Use of a marker of paragraph 19 or 20 for identification of increased Tobamovirus resistance in a plant.
22. Method for increasing Tobamovirus resistance in a plant, in particular CGMMV or ToBRFV resistance, comprising introducing a modification in a Tom2a gene, wherein the modification leads to a Tom2a gene as defined in any one of paragraphs 13-17.
23. Method for increasing Tobamovirus resistance in a plant, comprising introducing the modified Tom2a gene of any one of paragraphs 13-17 into a plant.
24. Method for producing a plant of the family Cucurbitaceae or Solanaceae that has increased Tobamovirus resistance, in particular increased CGMMV or ToBRFV resistance, comprising introducing a modification in a Tom2a gene comprising SEQ ID No. 1, SEQ ID No. 2, SEQ ID No. 3, SEQ ID No. 4, SEQ ID No. 5, or SEQ ID No. 6, or in another homologous sequence having at least 70% sequence identity to SEQ ID No. 1.
25. The method of paragraph 24 wherein the modification leads to a Tom2a gene as defined in any one of paragraphs 13-17.
26. Method for the production of a plant which has increased resistance to a Tobamovirus, in particular to CGMMV or ToBRFV, comprising:
   a) crossing the plant of any one of paragraphs 1-12, with another plant;
   b) optionally performing one or more rounds of selfing and/or crossing of the plant resulting from the cross of step a) to obtain a further generation population;
   c) selecting from the population resulting from the cross of step a), or from the further generation population of step b), a plant that comprises the modified Tom2a gene of any one of paragraphs 13-17.
27. Method for the production of a hybrid seed, comprising crossing a first parent plant with a second parent plant and harvesting the resultant hybrid seed, wherein the first parent plant and/or the second parent plant is the plant of any one of paragraphs 1-12, and wherein the hybrid plant that is grown from the seed has increased Tobamovirus resistance, in particular increased CGMMV or ToBRFV resistance.
28. Method of paragraph 27 for the production of a hybrid Cucumis sativus seed that has increased CGMMV resistance.
29. Method of paragraph 27 for the production of a hybrid Solanum lycopersicum seed that has increased ToBRFV resistance.
30. The hybrid seed produced by the method of any one of paragraphs 27-29.
31. Method for identification of a plant comprising a modified Tom2a gene of any one of paragraphs 13-17, wherein the identification comprises determining the presence of a modification in a Tom2a gene comprising SEQ ID No. 1, SEQ ID No. 2, SEQ ID No. 3, SEQ ID No. 4, SEQ ID No. 5, SEQ ID No. 6, or in another homologous sequence of SEQ ID No. 1, and optionally analyzing if the plant comprising the modification has increased resistance to a Tobamovirus, in particular to CGMMV or ToBRFV.
32. Propagation material suitable for producing the plant of any one of paragraphs 1-12, wherein the propagation material is suitable for sexual reproduction, and is in particular selected from a microspore, pollen, an ovary, an ovule, an embryo sac, and an egg cell; or is suitable for vegetative reproduction, and is in particular selected from a cutting, a root, a stem cell, and a protoplast; or is suitable for tissue culture of regenerable cells, and is in particular selected from a leaf, pollen, an embryo, a cotyledon, a hypocotyl, a meristematic cell, a root, a root tip, an anther, a flower, a seed, and a stem; wherein the plant produced from the propagation material comprises the modified Tom2a gene of any one of paragraphs 13-17 that provides increased resistance to a Tobamovirus, in particular to CGMMV or ToBRFV.
33. Method for seed production, comprising growing a plant from the seed of paragraph 18 that comprises the modified Tom2a gene of any one of paragraphs 13-17, allowing the plant to produce a fruit with seed, harvesting the fruit, and extracting those seed.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A plant comprising a modified Tom2a gene, the wildtype of which comprises SEQ ID No. 1, SEQ ID No. 2, SEQ ID No. 3, SEQ ID No. 4, SEQ ID No. 5, or SEQ ID No. 6, or comprises another homologous sequence having at least 70% sequence identity to SEQ ID No. 1, which modified gene leads to increased resistance to a Tobamovirus.

2. The plant of claim 1, which comprises a modification in the Tom2a gene that leads to a modified Tom2a protein that has a deletion, a substitution, or an insertion of at least one amino acid when compared to SEQ ID No. 7, SEQ ID No. 8, SEQ ID No. 9, SEQ ID No. 10, SEQ ID No. 11, or SEQ ID No. 12, or compared to another homologous sequence having at least 70% sequence similarity to SEQ ID No. 7.

3. The plant of claim 1, wherein the modification results in a premature stop codon which leads to a truncated protein, or wherein the Tom2a protein encoded by the modified Tom2a gene is absent, has a changed function, a reduced function, or is non-functional.

4. A Cucurbitaceae or Solanaceae plant comprising a modified Tom2a gene, wherein the Cucurbitaceae plant has resistance to Cucumber Green Mottle Mosaic Virus (CGMMV) and the Solanacae plant has resistance to Tomato Brown Rugose Fruit Virus (ToBRFV), and the modified Tom2a gene leads to:
a transmembrane domain modified Tom2a protein, or
a C-terminal tail truncated Tom2a protein, or
a Tom2a protein having modification in the Non-CP EC1 loop, or
a Tom2a protein having the Non-CP EC2 loop, or
a Tom2a protein consisting of amino acids 1-267 of SEQ ID No. 7 or corresponding amino acids of a homologous Tom2a protein sequence, or
a Tom2a protein having a modification in amino acids 268-279 of SEQ ID No. 7 or corresponding amino acids in a homologous Tom2a protein sequence, or
an absence of Tom2a protein, or
an absence of Tom2a protein function, or
a Tom2a protein having reduced function in comparison with Tom2a protein function from a Tom2a gene not having the modification,
wherein:
the wildtype Tom2a gene has a sequence which comprises SEQ ID No. 1, SEQ ID No. 2, SEQ ID No. 3, SEQ ID No. 4, SEQ ID No. 5, SEQ ID No. 6, or a homologous sequence having at least 95% sequence identity to SEQ ID No. 1, and
the wildtype Tom2a protein has a sequence which comprises SEQ ID No. 7, SEQ ID No. 8, SEQ ID No. 9, SEQ ID No. 10, SEQ ID No. 11, SEQ ID No. 12, or a sequence having at least 95% sequence similarity to SEQ ID No. 7.

5. The plant of claim 1, wherein the modification in the Tom2a protein is present in a transmembrane domain or in the C-terminal tail, or wherein the modification in the Tom2a protein is present in Non-CP EC1 or Non-CP EC2.

6. The plant of claim 4, which is a Cucumis sativus plant, Cucumis melo plant, Citrullus lanatus plant, Cucurbita pepo plant, Solanum lycopersicum plant, or Capsicum annuum plant.

7. The plant of claim 1, wherein the modified Tom2a protein is truncated and comprises only amino acids 1-267 of SEQ ID No. 7 or less, or the corresponding number of amino acids of a homologous Tom2a protein sequence, or the modified Tom2a protein comprises a modification in amino acids 268-279 of SEQ ID No. 7 that leads to a changed function, a reduced function, or a non-functional protein, or in the corresponding amino acids of a homologous Tom2a protein sequence having the sequence of SEQ ID No. 8, SEQ ID No. 9, SEQ ID No. 10, SEQ ID No. 11, or SEQ ID No. 12.

8. The plant of claim 1, wherein the modified Tom2a protein comprises SEQ ID No. 14.

9. The plant of claim 1, which is a Cucurbitaceae or Solanaceae plant.

10. The plant of claim 9, which is a Cucumis sativus plant, Cucumis melo plant, Citrullus lanatus plant, Cucurbita pepo plant, Solanum lycopersicum plant, or Capsicum annuum plant.

11. The plant of claim 9 which is a Cucurbitaceae plant having increased resistance to Cucumber Green Mottle Mosaic Virus (CGMMV) or a Solanacae plant having increased resistance to Tomato Brown Rugose Fruit Virus (ToBRFV).

12. Seed of, or that produces, the plant of claim 4.

13. A modified Tom2a gene encoding a Tom2a protein comprising a deletion, a substitution, or an insertion of at least one amino acid when compared to SEQ ID No. 7, SEQ ID No. 8, SEQ ID No. 9, SEQ ID No. 10, SEQ ID No. 11, or SEQ ID No. 12, or compared to another homologous sequence having at least 70% sequence similarity to SEQ ID No. 7.

14. The modified Tom2a gene of claim 13, which modified gene comprises a premature stop codon and which encodes a truncated Tom2a protein, or wherein the Tom2a protein encoded by the modified Tom2a gene is absent, has a changed function, a reduced function, or is non-functional.

15. Propagation material suitable from or for producing the plant of claim 4, wherein the propagation material is suitable for sexual reproduction, and comprises a microspore, pollen, an ovary, an ovule, an embryo sac, or an egg cell: or the propagation material is suitable for vegetative reproduction and comprises a cutting, a root, a stem cell, or a protoplast: or the propagation material is suitable for tissue culture of regenerable cells and comprises a leaf, pollen, an embryo, a cotyledon, a hypocotyl, a meristematic cell, a root, a root tip, an anther, a flower, a seed, or a stem; wherein the plant produced from the propagation material comprises the modified Tom2a gene.

16. The modified Tom2a gene of claim 13, wherein the encoded modified Tom2a protein is truncated and comprises only amino acids 1-267 of SEQ ID No. 7 or less, or the corresponding number of amino acids of a homologous Tom2a protein sequence, or the modified Tom2a protein comprises a modification in amino acids 268-279 of SEQ ID No. 7 that leads to a changed function, a reduced function, or a non-functional protein, or in the corresponding amino acids of a homologous Tom2a protein sequence having the sequence of SEQ ID No. 8, SEQ ID No. 9, SEQ ID No. 10, SEQ ID No. 11, or SEQ ID No. 12.

17. The modified Tom2a gene of claim 13, wherein the presence of the modified gene in a plant leads to increased Tobamovirus resistance.

18. Seed of or from the plant of claim 1, or comprising a modified Tom2a gene that leads to:
a transmembrane domain modified Tom2a protein, or
a C-terminal tail truncated Tom2a protein, or
a Tom2a protein having modification in the Non-CP EC1 loop, or
a Tom2a protein having the Non-CP EC2 loop, or
a Tom2a protein consisting of amino acids 1-267 of SEQ ID No. 7 or corresponding amino acids of a homologous Tom2a protein sequence, or
a Tom2a protein having a modification in amino acids 268-279 of SEQ ID No. 7 or corresponding amino acids in a homologous Tom2a protein sequence, or
an absence of Tom2a protein, or
an absence of Tom2a protein function, or
a Tom2a protein having reduced function in comparison with Tom2a protein function from a Tom2a gene not having the modification.

19. A method for identifying a plant having a modified Tom2a gene comprising genetic analysis of the plant using a marker having the sequence of any one of SEQ ID Nos. 15-24.

20. A method for producing a Cucurbitaceae plant having CGMMV resistance comprising:
a) crossing the Cucurbitaceae plant of claim 4 with another Cucurbitaceae plant;
b) optionally performing one or more rounds of selfing and/or crossing of the Cucurbitaceae plant resulting from the cross of step a) to obtain a further generation population;
c) selecting from the population resulting from the cross of step a), or from the further generation population of step b), a Cucurbitaceae plant that comprises the modified Tom2a gene or exhibits CGMMV resistance.

21. A method for producing a Solanacae plant having ToBRFV resistance comprising:
a) crossing the Solanacae plant of claim 4 with another Solanacae plant;
b) optionally performing one or more rounds of selfing and/or crossing of the Solanacae plant resulting from the cross of step a) to obtain a further generation population;
c) selecting from the population resulting from the cross of step a), or from the further generation population of step b), a Solanacae plant that comprises the modified Tom2a gene or exhibits ToBRFV resistance.

22. A method for increasing Tobamovirus resistance in a plant comprising introducing a modification in a Tom2a gene and thereby producing in the plant a modified Tom2a gene, or comprising introducing into the plant a modified Tom2a gene, wherein the modified Tom2a gene leads to:
a transmembrane domain modified Tom2a protein, or
a C-terminal tail truncated Tom2a protein, or
a Tom2a protein having modification in the Non-CP EC1 loop, or
a Tom2a protein having the Non-CP EC2 loop, or
a Tom2a protein consisting of amino acids 1-267 of SEQ ID No. 7 or corresponding amino acids of a homologous Tom2a protein sequence, or
a Tom2a protein having a modification in amino acids 268-279 of SEQ ID No. 7 or corresponding amino acids in a homologous Tom2a protein sequence, or
an absence of Tom2a protein, or
an absence of Tom2a protein function, or
a Tom2a protein having reduced function in comparison with Tom2a protein function from a Tom2a gene not having the modification.

23. Seed of claim 12, wherein the plant is a Cucumis sativus plant, Cucumis melo plant, Citrullus lanatus plant, Cucurbita pepo plant, Solanum lycopersicum plant, or Capsicum annuum plant.

24. A method for producing a Cucurbitaceae or Solanaceae plant that has increased Tobamovirus resistance comprising introducing a modification in a Tom2a gene comprising SEQ ID No. 1, SEQ ID No. 2, SEQ ID No. 3, SEQ ID No. 4, SEQ ID No. 5, or SEQ ID No. 6, or in another homologous sequence having at least 70% sequence identity to SEQ ID No. 1,
wherein the modified Tom2a gene leads to;
a transmembrane domain modified Tom2a protein, or
a C-terminal tail truncated Tom2a protein, or
a Tom2a protein having modification in the Non-CP EC1 loop, or
a Tom2a protein having the Non-CP EC2 loop, or
a Tom2a protein consisting of amino acids 1-267 of SEQ ID No. 7 or corresponding amino acids of a homologous Tom2a protein sequence, or
a Tom2a protein having a modification in amino acids 268-279 of SEQ ID No. 7 or corresponding amino acids in a homologous Tom2a protein sequence, or
an absence of Tom2a protein, or
an absence of Tom2a protein function, or
a Tom2a protein having reduced function in comparison with Tom2a protein function from a Tom2a gene not having the modification.

25. Propagation material of claim 15, wherein the plant is a Cucumis sativus plant, Cucumis melo plant, Citrullus lanatus plant, Cucurbita pepo plant, Solanum lycopersicum plant, or Capsicum annuum plant.

26. A method for the production of a plant which has increased resistance to a Tobamovirus comprising:
a) crossing the plant of claim 1, with another plant;
b) optionally performing one or more rounds of selfing and/or crossing of the plant resulting from the cross of step a) to obtain a further generation population;
c) selecting from the population resulting from the cross of step a), or from the further generation population of step b), a plant that comprises the modified Tom2a gene or exhibits Tobamovirus resistance.

27. A method for producing a hybrid seed, comprising crossing a first parent plant with a second parent plant and harvesting the resultant hybrid seed, wherein the first parent plant and/or the second parent plant is the plant of claim 1, and wherein the hybrid plant that is grown from the seed has the modified Tom2a gene and increased Tobamovirus resistance.

28. The method of claim 27, wherein the hybrid-seed is Cucumis sativus seed having increased CGMMV resistance, or the hybrid seed is Solanum lycopersicum seed having increased ToBRFV resistance.

29. A method for producing a hybrid Cucurbitaceae plant seed or hybrid Solanacae plant, comprising crossing a first Cucurbitaceae parent plant with a second Cucurbitaceae parent plant or crossing a first Solanacae parent plant with a second Solanacae parent plant and harvesting the resultant hybrid seed, wherein the first parent plant and/or the second parent plant is the Cucurbitaceae plant or the Solanacae plant of claim 4, and wherein the Cucurbitaceae hybrid plant grown from the seed has CGMMV resistance and the Solanacae hybrid plant grown from the seed has ToBRFV resistance.

30. The hybrid seed produced by the method of claim 27.

31. A method for identifying of a plant comprising a modified Tom2a gene that leads to:
a transmembrane domain modified Tom2a protein, or
a C-terminal tail truncated Tom2a protein, or
a Tom2a protein having modification in the Non-CP EC1 loop, or
a Tom2a protein having the Non-CP EC2 loop, or
a Tom2a protein consisting of amino acids 1-267 of SEQ ID No. 7 or corresponding amino acids of a homologous Tom2a protein sequence, or
a Tom2a protein having a modification in amino acids 268-279 of SEQ ID No. 7 or corresponding amino acids in a homologous Tom2a protein sequence, or
an absence of Tom2a protein, or
an absence of Tom2a protein function, or
a Tom2a protein having reduced function in comparison with Tom2a protein function from a Tom2a gene not having the modification,
the method comprising:
determining by genetic analysis modification in a Tom2a gene comprising SEQ ID No. 1, SEQ ID No. 2, SEQ ID No. 3, SEQ ID No. 4, SEQ ID No. 5, SEQ ID No. 6, or in another Tom2a gene having at least 95% sequence identity to the sequence of SEQ ID No. 1, and optionally analyzing if the plant comprising the modification has increased resistance to a Tobamovirus.

32. Propagation material suitable from or for producing the plant of claim 1, wherein the propagation material is suitable for sexual reproduction, and comprises a microspore, pollen, an ovary, an ovule, an embryo sac, or an egg cell; or the propagation material is suitable for vegetative reproduction and comprises a cutting, a root, a stem cell, or a protoplast; or the propagation material is suitable for tissue culture of regenerable cells and comprises a leaf, pollen, an embryo, a cotyledon, a hypocotyl, a meristematic cell, a root, a root tip, an anther, a flower, a seed, or a stem; wherein the plant produced from the propagation material comprises the modified Tom2a gene that provides increased resistance to a Tobamovirus, and that leads to
a transmembrane domain modified Tom2a protein, or
a C-terminal tail truncated Tom2a protein, or
a Tom2a protein having modification in the Non-CP EC1 loop, or
a Tom2a protein having the Non-CP EC2 loop, or
a Tom2a protein consisting of amino acids 1-267 of SEQ ID No. 7 or corresponding amino acids of a homologous Tom2a protein sequence, or
a Tom2a protein having a modification in amino acids 268-279 of SEQ ID No. 7 or corresponding amino acids in a homologous Tom2a protein sequence, or
an absence of Tom2a protein, or
an absence of Tom2a protein function, or
a Tom2a protein having reduced function in comparison with Tom2a protein function from a Tom2a gene not having the modification.

33. A method for producing seed comprising growing a plant from the seed of claim 18, allowing the plant to produce a fruit with seed, harvesting the fruit, and extracting these seed from the fruit.

34. The plant of claim 10, which is a Cucumis sativus plant or Solanum lycopersicum plant.

35. The modified Tom2a gene of claim 17, wherein the plant is a Cucurbitaceae plant and the Tobamovirus is CGMMV, or the plant is a Solanaceae plant and the Tobamovirus is ToBRFV.

36. The method of claim 29, wherein the Cucurbitaceae plant is a Cucumis sativus plant, Cucumis melo plant, Citrullus lanatus plant, or Cucurbita pepo plant, and the Solanacae plant is Solanum lycopersicum plant or Capsicum annuum plant.

37. The method of claim 22, wherein the Tobamovirus is CGMMV or ToBRFV.

38. The method of claim 20, wherein the Cucurbitaceae plant is a Cucumis sativus plant, Cucumis melo plant, Citrullus lanatus plant.

39. The method of claim 24, wherein the plant is a Cucurbitaceae plant and the Tobamovirus is CGMMV or the plant is a Solanaceae plant and the Tobamovirus is ToBRFV.

40. The method of claim 21, wherein the Solanacae plant is Solanum lycopersicum plant or Capsicum annuum plant.

41. The method of claim 26, wherein the plant is a Cucurbitaceae plant and the Tobamovirus is CGMMV or the plant is a Solanaceae plant and the Tobamovirus is ToBRFV.

42. The plant of claim 4, which is a Cucumis sativus plant or a Solanum lycopersicum plant.

43. The method of claim 27, wherein the hybrid plant that is grown from the seed is a Cucurbitaceae-plant and the Tobamovirus is CGMMV or the plant is a Solanaceae plant and the Tobamovirus is ToBRFV.

44. Seed of claim 12 which is Cucumis sativus seed or Solanum lycopersicum seed.

45. The method of claim 31, wherein the Tobamovirus is CGMMV or ToBRFV.

46. The method of claim 45, including performing the analyzing step.

47. A Cucurbitaceae-plant having CGMMV resistance, or a Solanaceae plant and the having ToBRFV produced from the propagation material of claim 32.

48. Propagation material of claim 15, wherein the plant is Cucumis sativus plant or Solanum lycopersicum plant.


SEQUENCE LISTING (26 sequences)

SEQ ID NO: 1 - DNA, 840 bp, Cucumis sativus
  misc_feature 1..840: note = CDS (Csa3G904130.1)
  source 1..840: mol_type = genomic DNA
atggcctgcagagggtgctttgagtgcctattgaagcttttgaacttcttcttgtccctgctgggtcttgccatggtgggctatgggatttacttattggttgagtacttgcaatctcatagtgatgttccaggaccttcgttgagtggtgatcatgatctggtccagcttggtcgaccaatgctaatggctgtgtctctgtcttctagcatctttgacaaccttccaaaagcctggttcatatatttgttcattgctacgggagtcactatctttgttgtctcttgttttggctgtattggagctgcaacacgtagtggatgctgtttaagttgttattcgattttattgcttctactgattttggttcaactaggatgtggtgccttcatattctttgacaaaaattggagagatgaaattcctggggacagaacaggaaactttgataagatctatgaacttctggaaagcaagtgggaaatcatcagatgggttgcactaggaactataatttttgaggctctccttttcttgttggctcttgtggttcgtgcagcaaacagacctgtagactatgacagtgatgatgaatacattgctccaaggcaacaaatccgacaaccgttgatcaataggcccgttgctccagcaacaggtgtgcctgttgctgggacacttgatcaacgaccaagtcgaaatgatgcttggagtacacgaatgagggaaaagtatgggctggatacttcggagttcacgtacaacccatctgagtctcacaggtttcagcaagttgctcctcagccagccgaagaaaagagccgctgcacaatcatgtga

SEQ ID NO: 2 - DNA, 840 bp, Cucumis melo
  misc_feature 1..840: note = CDS (MELO3C003376)
  source 1..840: mol_type = genomic DNA
atggcctgcagagggtgctttgagtgcctattgaagcttttgaacttcttcttatctctgctgggtcttgccatggtgggctatgggatttacttgttggttgagtacttgcaatcttctagtgatgttccaggaccttccttgagtggtgatcatgatctggtccagcttggtcgaccaatgctaatggccgtgtctctgtcttctaacatctttgataaccttccaaaagcctggttcatatatttgttcattgctacgggagtcattgtctttgttgtctcttgttttggctgtattggagctgcaacacgtagtggatgctgtttaagttgttattcgattttattgcttctactgattttggtgcaactaggatgtgctgccttcatattctttgacaaaaattggagagatgaaattcctggggacaaaacaggaaactttgataagatctatgaacttctggagaacaagtggaaaatcatcagatgggttgcattaggaactataatttttgaggctctccttttcttgttggctcttgtggttcgtgcagcaaacagacctgtagactatgacagtgatgatgagtacattgctccaaggcaacaaatccgacaaccgttgatcaataggcccgttgctccagcaactggtgtgcctgttgctgggacacttgatcaacgaccaagtcgaaatgatgcttggagtgcacgaatgagggaaaagtatgggctggatacttcggagttcacgtacaacccatccgagtctcacaggtttcagcaagttgctcctcaggcagccgaagaaaagagccgctgcaccatcatgtga

SEQ ID NO: 3 - DNA, 840 bp, Citrullus lanatus
  misc_feature 1..840: note = CDS (Cla97C09G175160)
  source 1..840: mol_type = genomic DNA
atggcctgcagagggtgctttgagtgcctattgaagcttctgaacttcttcttatccctgctgggtcttgccatggtgggctatgggatttacttgttggttgagtatttgcaatctcctagtgatgtcccaggaccttcgtcgagtggtgatcatgatctggtccagcttggtcgaccaatgctaatggccgtgtctctgtcttctaatatctttgataaccttccaaaagcctggttcatatacttgttcattgctgtgggagtcattatctttgttgtctcttgttttgggtgtattggagctgcaacacgtaatggatgctgtttaagttgttattcagttttgctgcttctactgattttggtgcaactaggatgtgctgccttcatattctttgacaaaacttggagagatgaaattcctggggacaaaacaggaaactttgataagatctatgaactccttgaagacaagtgggaaatcatcagatgggttgcattgggagctgtaatttttgaggccctccttttcttgttggctcttgtggttcgtgcagcaaacagacctgcagactatgacagcgatgatgagtacattgctccaaggcaacaaatcagacaaccattgatcaataggcctgctgctcctgcaacaggtgtgcctgtggctgggacacttgatcaacgaccaagtcgaaatgatgcttggagtacacgaatgagggaaaagtatgggctggatacttcagagttcacatacaacccatctgagtctcacaggtttcagcaagttgcccctcagccagccgaagaaaagagccgctgcaccatcatgtga

SEQ ID NO: 4 - DNA, 840 bp, Cucurbita pepo
  misc_feature 1..840: note = CDS (Cp4.1LG05g01990)
  source 1..840: mol_type = genomic DNA
atggcctgcagagggtgctttgagtgcctattgaagcttctgaacttcttcttatccctgctgggtcttgccatggtgggctatgggatttacttgttagttgagtacttgcaatcttctagtgatgttccaagacctccgttgagtggtgatcatgatgtgattcagcttggtcgaccaatgctaatggccgtgtctctgtcttctaacatctttgataaccttccaagagcctggttcatatacttgttcattgctgtgggagtcattatctttgttgtctcttgttttgggtgtattggagctgccacacgtaatggatgctgtttaagttgttattcagttttggtgcttctactgattttggtgcaactaggatgtgcagcattcatattctttgacaaacattggagagatgaaattcctggggatagaactgggaactttgataaaatctatgaactcctggaagacaagtgggaaatcatcagatgggttgcattaggagctgtaatttttgaggctctccttttcttgttggcccttgtggttcgtgcagcaaacagacctttagactatgacagtgatgatgagtacattgctccaaggcaacaaatccgacaaccgttgatcaataggcctgttgctcctgcaactggtgtgcctgttgctgggacacttgatcaacgaccaagccgaaatgatgcttggagtacacgaatgagggaaaagtatgggctggatacttccgagttcacatacaacccatctgagtctcacaggtttcagcaagttgccccacagccagcagaagaaaagagccgctgcaccatcatgtga

SEQ ID NO: 5 - DNA, 855 bp, Solanum lycopersicum
  misc_feature 1..855: note = CDS (Solyc08g077220.2.1)
  source 1..855: mol_type = genomic DNA
atggcgtgcaaagggttttgggagtgcttgttgaagctcttgaactttttgttgacccttgttggtttgacaatggtggggtatggtatttatctatttgttgagtacaaaaatcattcacactccggagatgattacccagttgcaccacctatgagtggtgacatgatagagtttggtcgtccaatgctgatggctgtatcgttggctgaaaacatatttgataaacttccaaaaccttggttcatatatttgtttattggtattggagcagttcttgtagttgtatcttgctgtggttgtattggagcggcaacaaggaatggttgctgcctgagttgttactccatgttgattttcttgttgatcttggtagagctaggtgctgctggttttatattctttgataaaagctggaaagatgaaattccaagggataaaacgggcaactttgaaacgatctatgactttctggatgaccactggaagattattaagtgggttgcccttggtgctgttatattcgaggctcttatattcttattggccctcgtagtaagggcagcaaacagaccagcagactatgatagtgatgatgagtacataggtggtcccagacaacaaatccgacagccactgatcaacaataggccaccagcaaatcctgcaactggtgtccctgttactgctactcttgataatcgtccaagtagaaatgatgcgtggagtacacgtatgagggaaaagtatggacttgacacatcagagtttacttacaacccatcggagtcgaacagatatccgccaactgccgcacagccgcaagaggaaaggaagggttgtaccataatgtga

SEQ ID NO: 6 - DNA, 858 bp, Capsicum annuum
  misc_feature 1..858: note = CDS (XP_016571245.1)
  source 1..858: mol_type = genomic DNA
atggcgtgcaaaggtttttgggagtgtttgttgaagctcttgaacttcttgttgacccttgttggtttggcaatggtggggtatggtatttatctatttgttgaatacaaaaatcattcatcctccggggatgatcaccctgttgcaccacctacaagtgctgctgatgtgatagagtttggtcgtccaatgcttatggctgtatcgttggctgataacatctttgataaacttccgaaaccttggttcatatatttgtttattggtgttggagcagttcttgtaattgtgtcttgctgtggttgcattggagcggcaacaaggaacggctgttgcctgagttgttactccgtgttgattttcttgttgatcctggtagagctaggtgctgctggttttatgttctttgataaaagctggagagaggaaattccgactgacaaaacgggtaactttgatacgatctatggatttttggatgaacactggaagattgtcaagtgggttgcccttggtgctgttattttagaggctcttatattcttattggccctcatagtaagggcggcaaacagaccagcagactatgatagtgatgatgagtacataggtggtcccagacaacaaatccgacagccactgatcagcaatagggcaccaccaaatgctgcaactggtgtccctgttactggtacccttgataatcgtccaagtagaaatgacgcatggagtacacgtatgagagaaaagtatgggctcgatacatcggagttcacttacaacccgtcagagtccaacagatatccacaaacagccgtacagccgcaagaggaaaagaagggttgtgccataatgtga

SEQ ID NO: 7 - protein, 279 aa, Cucumis sativus
  REGION 1..279: note = protein (Csa3G904130.1)
MACRGCFECLLKLLNFFLSLLGLAMVGYGIYLLVEYLQSHSDVPGPSLSGDHDLVQLGRPMLMAVSLSSSIFDNLPKAWFIYLFIATGVTIFVVSCFGCIGAATRSGCCLSCYSILLLLLILVQLGCGAFIFFDKNWRDEIPGDRTGNFDKIYELLESKWEIIRWVALGTIIFEALLFLLALVVRAANRPVDYDSDDEYIAPRQQIRQPLINRPVAPATGVPVAGTLDQRPSRNDAWSTRMREKYGLDTSEFTYNPSESHRFQQVAPQPAEEKSRCTIM

SEQ ID NO: 8 - protein, 279 aa, Cucumis melo
  REGION 1..279: note = protein (MELO3C003376)
MACRGCFECLLKLLNFFLSLLGLAMVGYGIYLLVEYLQSSSDVPGPSLSGDHDLVQLGRPMLMAVSLSSNIFDNLPKAWFIYLFIATGVIVFVVSCFGCIGAATRSGCCLSCYSILLLLLILVQLGCAAFIFFDKNWRDEIPGDKTGNFDKIYELLENKWKIIRWVALGTIIFEALLFLLALVVRAANRPVDYDSDDEYIAPRQQIRQPLINRPVAPATGVPVAGTLDQRPSRNDAWSARMREKYGLDTSEFTYNPSESHRFQQVAPQAAEEKSRCTIM

SEQ ID NO: 9 - protein, 279 aa, Citrullus lanatus
  REGION 1..279: note = protein (Cla97C09G175160)
MACRGCFECLLKLLNFFLSLLGLAMVGYGIYLLVEYLQSPSDVPGPSSSGDHDLVQLGRPMLMAVSLSSNIFDNLPKAWFIYLFIAVGVIIFVVSCFGCIGAATRNGCCLSCYSVLLLLLILVQLGCAAFIFFDKTWRDEIPGDKTGNFDKIYELLEDKWEIIRWVALGAVIFEALLFLLALVVRAANRPADYDSDDEYIAPRQQIRQPLINRPAAPATGVPVAGTLDQRPSRNDAWSTRMREKYGLDTSEFTYNPSESHRFQQVAPQPAEEKSRCTIM

SEQ ID NO: 10 - protein, 279 aa, Cucurbita pepo
  REGION 1..279: note = protein (Cp4.1LG05g01990)
MACRGCFECLLKLLNFFLSLLGLAMVGYGIYLLVEYLQSSSDVPRPPLSGDHDVIQLGRPMLMAVSLSSNIFDNLPRAWFIYLFIAVGVIIFVVSCFGCIGAATRNGCCLSCYSVLVLLLILVQLGCAAFIFFDKHWRDEIPGDRTGNFDKIYELLEDKWEIIRWVALGAVIFEALLFLLALVVRAANRPLDYDSDDEYIAPRQQIRQPLINRPVAPATGVPVAGTLDQRPSRNDAWSTRMREKYGLDTSEFTYNPSESHRFQQVAPQPAEEKSRCTIM

SEQ ID NO: 11 - protein, 284 aa, Solanum lycopersicum
  REGION 1..284: note = protein (Solyc08g077220.2.1)
MACKGFWECLLKLLNFLLTLVGLTMVGYGIYLFVEYKNHSHSGDDYPVAPPMSGDMIEFGRPMLMAVSLAENIFDKLPKPWFIYLFIGIGAVLVVVSCCGCIGAATRNGCCLSCYSMLIFLLILVELGAAGFIFFDKSWKDEIPRDKTGNFETIYDFLDDHWKIIKWVALGAVIFEALIFLLALVVRAANRPADYDSDDEYIGGPRQQIRQPLINNRPPANPATGVPVTATLDNRPSRNDAWSTRMREKYGLDTSEFTYNPSESNRYPPTAAQPQEERKGCTIM

SEQ ID NO: 12 - protein, 285 aa, Capsicum annuum
  REGION 1..285: note = protein (XP_016571245.1)
MACKGFWECLLKLLNFLLTLVGLAMVGYGIYLFVEYKNHSSSGDDHPVAPPTSAADVIEFGRPMLMAVSLADNIFDKLPKPWFIYLFIGVGAVLVIVSCCGCIGAATRNGCCLSCYSVLIFLLILVELGAAGFMFFDKSWREEIPTDKTGNFDTIYGFLDEHWKIVKWVALGAVILEALIFLLALIVRAANRPADYDSDDEYIGGPRQQIRQPLISNRAPPNAATGVPVTGTLDNRPSRNDAWSTRMREKYGLDTSEFTYNPSESNRYPQTAVQPQEEKKGCAIM

SEQ ID NO: 13 - DNA, 804 bp, Cucumis sativus
  misc_feature 1..804: note = Tom2a CDS (mutant nr 2720)
  source 1..804: mol_type = genomic DNA
atggcctgcagagggtgctttgagtgcctattgaagcttttgaacttcttcttgtccctgctgggtcttgccatggtgggctatgggatttacttattggttgagtacttgcaatctcatagtgatgttccaggaccttcgttgagtggtgatcatgatctggtccagcttggtcgaccaatgctaatggctgtgtctctgtcttctagcatctttgacaaccttccaaaagcctggttcatatatttgttcattgctacgggagtcactatctttgttgtctcttgttttggctgtattggagctgcaacacgtagtggatgctgtttaagttgttattcgattttattgcttctactgattttggttcaactaggatgtggtgccttcatattctttgacaaaaattggagagatgaaattcctggggacagaacaggaaactttgataagatctatgaacttctggaaagcaagtgggaaatcatcagatgggttgcactaggaactataatttttgaggctctccttttcttgttggctcttgtggttcgtgcagcaaacagacctgtagactatgacagtgatgatgaatacattgctccaaggcaacaaatccgacaaccgttgatcaataggcccgttgctccagcaacaggtgtgcctgttgctgggacacttgatcaacgaccaagtcgaaatgatgcttggagtacacgaatgagggaaaagtatgggctggatacttcggagttcacgtacaacccatctgagtctcacaggtttcagcaagttgctccttag

SEQ ID NO: 14 - protein, 267 aa, Cucumis sativus
  REGION 1..267: note = Tom2a protein (mutant nr 2720
MACRGCFECLLKLLNFFLSLLGLAMVGYGIYLLVEYLQSHSDVPGPSLSGDHDLVQLGRPMLMAVSLSSSIFDNLPKAWFIYLFIATGVTIFVVSCFGCIGAATRSGCCLSCYSILLLLLILVQLGCGAFIFFDKNWRDEIPGDRTGNFDKIYELLESKWEIIRWVALGTIIFEALLFLLALVVRAANRPVDYDSDDEYIAPRQQIRQPLINRPVAPATGVPVAGTLDQRPSRNDAWSTRMREKYGLDTSEFTYNPSESHRFQQVAP

SEQ ID NO: 15 - DNA, 236 bp, Cucumis sativus
  misc_feature 1..236: note = Marker sequence
  source 1..236: mol_type = genomic DNA
gatctgatggcatgttcttttcacaatttttttcgagtctgactttgttctttttatttttgaaacagtatgggctggatacttcggagttcacgtacaacccatctgagtctcacaggtttcagcaagttgctccttagccagccgaagaaaagagccgctgcacaatcatgtgataaaagtatgccttgtactatttgtttctcactccactgtagtgttttagcgggtccgaa

SEQ ID NO: 16 - DNA, 40 bp, Cucumis sativus
  misc_feature 1..40: note = Marker sequence
  source 1..40: mol_type = genomic DNA
tgttttggctgtattggagcaacacgtagtggatgctgtt

SEQ ID NO: 17 - DNA, 41 bp, Cucumis sativus
  misc_feature 1..41: note = Marker sequence
  source 1..41: mol_type = genomic DNA
tcttgttggctcttgtggttggtgcagcaaacagacctgta

SEQ ID NO: 18 - DNA, 41 bp, Cucumis sativus
  misc_feature 1..41: note = Marker sequence
  source 1..41: mol_type = genomic DNA
ggcccgttgctccagcaacaagtgtgcctgttgctgggaca

SEQ ID NO: 19 - DNA, 42 bp, Cucumis sativus
  misc_feature 1..42: note = Marker sequence
  source 1..42: mol_type = genomic DNA
cctcagccagccgaagaaaagagccgctgcgcaatcatgtga

SEQ ID NO: 20 - DNA, 41 bp, Cucumis sativus
  source 1..41: mol_type = genomic DNA
cttcctttcctcttgcggctatgcggcagttggcggatatc

SEQ ID NO: 21 - DNA, 41 bp, Cucumis sativus
  source 1..41: mol_type = genomic DNA
ggcagcaaccattccttgttgctccaatacaaccacagcaa

SEQ ID NO: 22 - DNA, 41 bp, Cucumis sativus
  source 1..41: mol_type = genomic DNA
tgctggtctgtttgctgcccctactacgagggccaataaga

SEQ ID NO: 23 - DNA, 41 bp, Cucumis sativus
  source 1..41: mol_type = genomic DNA
accatatcatcacattatggcacaacccttcctttcctctt

SEQ ID NO: 24 - DNA, 41 bp, Cucumis sativus
  source 1..41: mol_type = genomic DNA
agtagcagtaacagggacactagttgcaggatttgctggtg

SEQ ID NO: 25 - DNA, 20 bp, Solanum lycopersicum
  source 1..20: mol_type = genomic DNA
tgcgtggagtacacgtatga

SEQ ID NO: 26 - DNA, 20 bp, Solanum lycopersicum
  source 1..20: mol_type = genomic DNA
ccagttgcaccacctatgag